(19) United States
(12) Patent Application Publication
(10) Pub. No.: US 2025/0213142 A1
(43) Pub. Date: Jul. 3, 2025

(54) Audioplethysmography and Motion-Sensing Data Fusion

(71) Applicant: Google LLC, Mountain View, CA (US)

(72) Inventors: Patrick M. Amihood, Palo Alto, CA (US); Xiaoran Fan, Irvine, CA (US); Cody Wortham, San Francisco, CA (US)

(73) Assignee: Google LLC, Mountain View, CA (US)

(21) Appl. No.: 18/400,834

(22) Filed: Dec. 29, 2023

(51) Int. Cl. A61B 5/12; A61B 5/00

(52) U.S. Cl. CPC A61B 5/125; A61B 5/721

(57) ABSTRACT

Techniques and apparatuses are described that perform audioplethysmography and motion-sensing data fusion. Fusing motion-sensing data with audioplethysmography expands the situations in which audioplethysmography can operate. In one aspect, audioplethysmography and motion-sensing data fusion can be used for motion-artifact filtering. With motion-artifact filtering, noise caused by motion of a user can be attenuated to improve sensitivity and accuracy for audioplethysmography. This performance improvement expands the ability of audioplethysmography to support use cases during situations in which the user engages in an activity. In another aspect, audioplethysmography and motion-sensing data fusion can expand a functionality of a hearable to include activity detection and/or activity classification. While activity detection and/or activity classification can provide additional contextual information for other use cases associated with audioplethysmography, either can also be used to control an operation of the hearable and/or a computing device.

### BACKGROUND

Technological advances in medicine and healthcare are making it possible for people to live longer, healthier lives. To further achieve this, individuals have become interested in tracking their personal health. Health monitoring can motivate an individual to realize a particular fitness goal by tracking incremental improvements in the performance of the body's functions. Additionally, the individual can monitor the impact of various chronic illnesses on their body. With active feedback through health monitoring, the individual can live an active and full life with many chronic illnesses and quickly recognize situations in which it is necessary to seek medical attention.

Some devices that support health monitoring, however, can be obtrusive and uncomfortable. As such, people may choose to forego health monitoring if the device negatively impacts their movement or causes inconveniences while performing daily activities. It is therefore desirable for health-monitoring devices to be reliable, portable, and affordable to encourage more users to take advantage of these features.

### SUMMARY

Techniques and apparatuses are described for audioplethysmography and motion-sensing data fusion. Fusing motion-sensing data with audioplethysmography expands the situations in which audioplethysmography can operate. In one aspect, audioplethysmography and motion-sensing data fusion can be used for motion-artifact filtering. With motion-artifact filtering, noise caused by motion of a user can be attenuated to improve sensitivity and accuracy for audioplethysmography. This performance improvement expands the ability of audioplethysmography to support use cases during situations in which the user engages in an activity. In another aspect, audioplethysmography and motion-sensing data fusion can expand a functionality of the hearable to include activity detection and/or activity classification. While activity detection and/or activity classification can provide additional contextual information for other use cases associated with audioplethysmography, either can also be used to control an operation of the hearable and/or a computing device.

Aspects described below include a method for performing audioplethysmography and motion-sensing data fusion. The method includes transmitting and receiving, during a first time period, an acoustic signal that propagates within at least a portion of an ear canal of a user. The received acoustic signal represents a version of the transmitted acoustic signal with one or more characteristics modified based on the propagation within the ear canal. The received acoustic signal includes at least one motion artifact associated with the user moving during at least a portion of the first time period. The method also includes accepting motion-sensing data generated by a motion sensor during the first time period. The method additionally includes processing a version of the received acoustic signal based on the motion-sensing data. The method further includes generating, based on the processing, data associated with at least one of the following: the one or more characteristics associated with the propagation of the acoustic signal within the ear canal or the at least one motion artifact associated with the user moving. In an example, the version of the received acoustic signal can represent an electrical version of the received acoustic signal, a digital version of the acoustic signal, and/or a downconverted version of the received acoustic signal.

Aspects described below include a computer-readable storage medium comprising instructions that, responsive execution by a processor, cause a hearable to perform any of the methods described herein.

Aspects described below include a device with at least one transducer and at least one processor. The device is configured to perform, using the at least one transducer and the at least one processor, any of the methods described herein.

Aspects described below include a system n with means for performing audioplethysmography and motion-sensing data fusion.

### BRIEF DESCRIPTION OF DRAWINGS

Apparatuses for and techniques that perform audioplethysmography and motion-sensing data fusion are described with reference to the following drawings. The same numbers are used throughout the drawings to reference like features and components:

FIG. 1-1 illustrates an example environment in which audioplethysmography can be implemented:

FIG. 1-2 illustrates an example geometric change in an ear canal, which can be detected using audioplethysmography;

FIG. 2 illustrates example environments in which audioplethysmography and motion-sensing data fusion can be implemented:

FIG. 3 illustrates an impact of motion on audioplethysmography:

FIG. 4 illustrates example components of a computing device:

FIG. 5 illustrates example components of a hearable:

FIG. 6 illustrates example operations of two hearables:

FIG. 7 illustrates an example implementation of a hearable capable of performing audioplethysmography and motion-sensing data fusion:

FIG. 8 illustrate an example flow diagram for operating a hearable:

FIG. 9 illustrates an example scheme implemented by a calibration module of a hearable:

FIG. 10 illustrates an example scheme implemented by a hearable to perform audioplethysmography and motion-sensing data fusion:

FIG. 11-1 illustrates a first example implementation of a motion-artifact filter for performing audioplethysmography and motion-sensing data fusion:

FIG. 11-2 illustrates a second example implementation of a motion-artifact filter for performing audioplethysmography and motion-sensing data fusion:

FIG. 12 illustrates an example implementation of a measurement module for performing audioplethysmography and motion-sensing data fusion:

FIG. 13 illustrates an example method for performing an aspect of audioplethysmography and motion-sensing data fusion:

FIG. 14 illustrates another example method for performing an aspect of audioplethysmography and motion-sensing data fusion:

FIG. 15 illustrate yet another example method for performing an aspect of audioplethysmography and motion-sensing data fusion; and

FIG. 16 illustrates an example computing system embodying, or in which techniques may be implemented that enable use of, audioplethysmography and motion-sensing data fusion.

### DETAILED DESCRIPTION

Technological advances in medicine and healthcare are making it possible for people to live longer, healthier lives. To further achieve this, individuals have become interested in tracking their personal health. Health monitoring can motivate an individual to realize a particular fitness goal by tracking incremental improvements in the performance of the body's functions. Additionally, the individual can use health monitoring to observe changes in the body caused by chronic illnesses. With active feedback through health monitoring, the individual can live an active and full life with many chronic illnesses and recognize situations in which it is necessary to quickly seek medical attention.

Some health-monitoring devices, however, can be obtrusive and uncomfortable. To measure carbon dioxide levels, for example, some devices take a sample of blood from the user. Other devices may utilize auxiliary sensors, including optical or electronic sensors, that add additional weight, cost, complexity, and/or bulk. Still other devices may require constant recharging of a battery due to relatively high power usage. As such, people may choose to forego health monitoring if the health-monitoring device negatively impacts their movement or causes inconveniences while performing daily activities. It is therefore desirable for health-monitoring devices to be reliable, portable, efficient, and affordable to expand accessibility to more users.

Wireless technology has become prevalent in everyday life, making communication and data readily accessible to users. One type of wireless technology are wireless hearables, examples of which include wireless earbuds and wireless headphones. Wireless hearables have allowed users freedom of movement while listening to audio content from music, audio books, podcasts, and videos. With the prevalence of wireless hearables, there is a market for adding additional features to existing hearables utilizing current hardware (e.g., without introducing any new hardware).

Provided according to one or more preferred embodiments is a hearable, such as an earbud, that is capable of performing a novel physiological monitoring process termed herein audioplethysmography. Audioplethysmography is an active acoustic method capable of sensing subtle physiologically-related changes observable at a user's outer and middle ear. Instead of relying on other auxiliary sensors, such as optical or electrical sensors, audioplethysmography involves transmitting and receiving acoustic signals that at least partially propagate within a user's ear canal. To effectively perform audioplethysmography, the hearable should form at least a partial seal in or around the user's outer ear. Such a seal enables formation of an acoustic circuit, which includes the seal, the hearable, the ear canal, and an ear drum of the ear. By transmitting and receiving acoustic signals, the hearable can recognize changes in the acoustic circuit to monitor a user's biometrics, including heart rate, respiration rate, heart rate variability, and/or blood pressure. Audioplethysmography can also be used for other use cases, including speech detection, chewing detection, gesture recognition, and so forth. In addition to being relatively unobtrusive, some hearables can be configured to support audioplethysmography without the need for additional hardware. As such, the size, cost, and power usage of the hearable can help make health monitoring accessible to a larger group of people and improve the user experience with hearables.

While audioplethysmography can support a variety of different use cases, it can be sensitive to motion. As the user performs different activities, movement of the user's head can introduce motion artifacts (e.g., noise) within the received acoustic signals. A motion artifact represents a portion of the received acoustic signal where an amplitude, phase, and/or frequency is affected due to the user's movement. In one aspect, these motion artifacts can make it challenging for audioplethysmography to process the received acoustic signals and extract the desired information for a given use case. The motion artifacts can also decrease measurement accuracy and/or cause false detections.

The challenges of performing audioplethysmography in the presence of motion can differ from the motion-based challenges experienced by other types of sensors. As audioplethysmography is typically performed using hearables, audioplethysmography is particularly sensitive to the user's head movements. Generally speaking, a user may be more likely to move their head in more situations compared to other body parts. These situations can include those in which the user is relatively inactive (e.g., sitting, reading, or working at a desk). In contrast, other sensors that are positioned on the user's torso or appendage (e.g., wrist) may be subjected to less motion while the user is relatively inactive. Other activities, such as running, can subject audioplethysmography and these other sensors to different types of motion. As such, audioplethysmography can have different motion artifacts and challenges compared to other types of sensors.

To address this challenge and provide new features for existing hearables, techniques are described for audioplethysmography and motion-sensing data fusion. Fusing motion-sensing data with audioplethysmography expands the situations in which audioplethysmography can operate. In one aspect, audioplethysmography and motion-sensing data fusion can be used for motion-artifact filtering. With motion-artifact filtering, noise caused by motion of a user can be attenuated to improve sensitivity and accuracy for audioplethysmography. This performance improvement expands the ability of audioplethysmography to support use cases during situations in which the user engages in an activity. One such use case includes biometric monitoring, which can be performed using audioplethysmography while the user exercises.

In another aspect, audioplethysmography and motion-sensing data fusion can expand a functionality of the hearable to include activity detection and/or activity classification. Activity detection and/or activity classification can provide additional contextual information for other use cases associated with audioplethysmography. Additionally or alternatively, activity detection and/or activity classification can be used to control an operation of the hearable and/or a computing device.

### Operating Environment

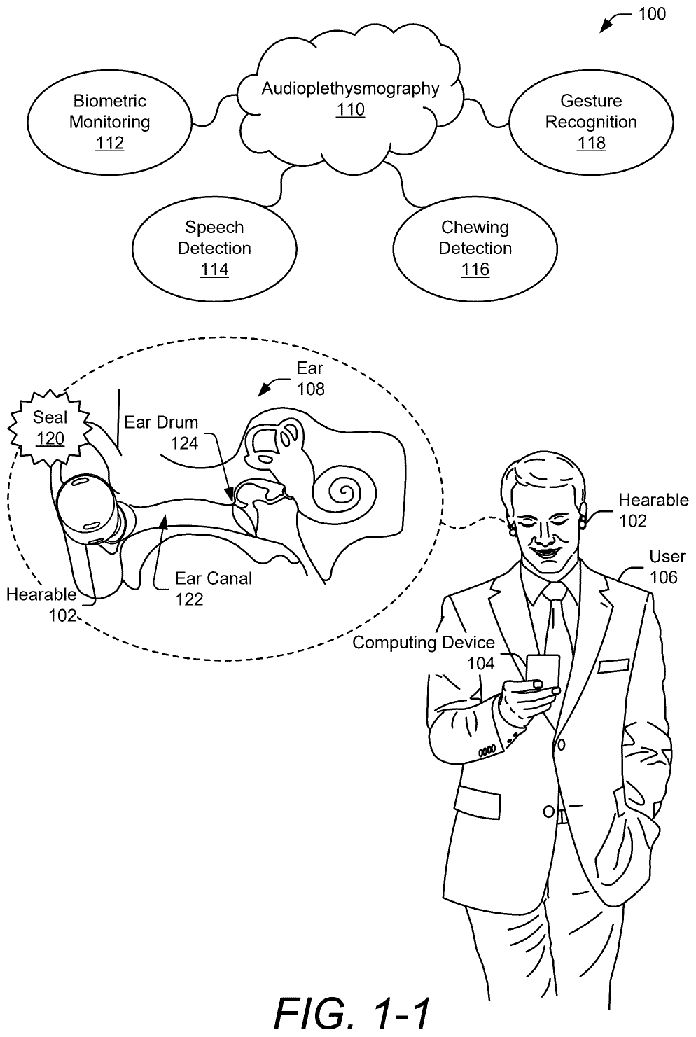

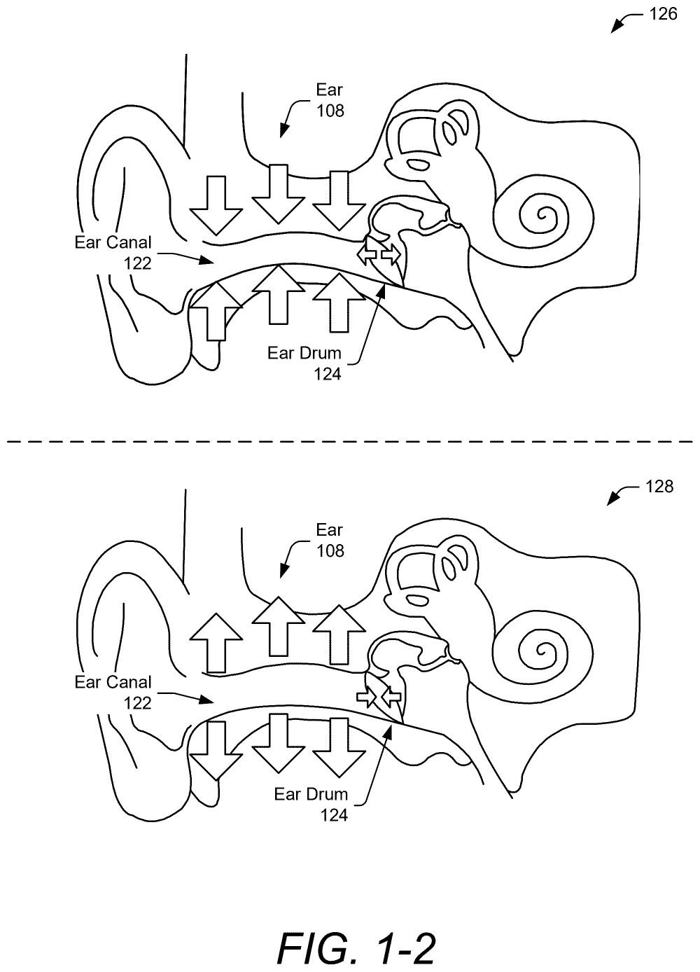

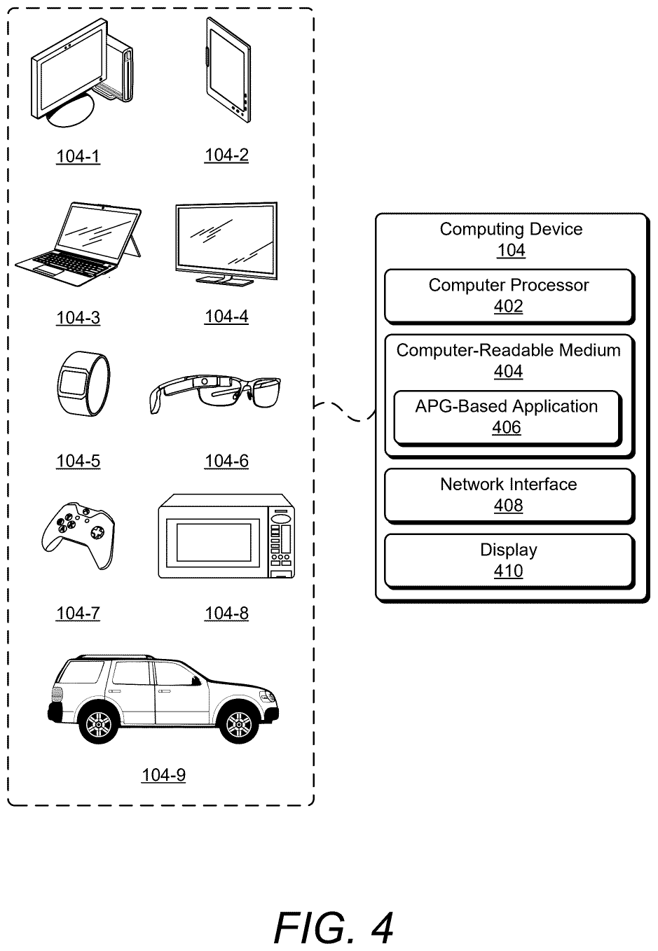

FIG. 1-1 is an illustration of an example environment 100 in which audioplethysmography can be implemented. In the example environment 100, a hearable 102 is connected to a computing device 104 using a physical or wireless interface. The hearable 102 is a device that can play audible content provided by the computing device 104 and direct the audible content into a user 106's ear 108. In this example, the hearable 102 operates together with the computing device 104. In other examples, the hearable 102 can operate or be implemented as a stand-alone device. Although depicted as a smartphone, the computing device 104 can include other types of devices, including those described with respect to FIG. 4.

The hearable 102 is capable of performing audioplethysmography 110, which is an acoustic method of sensing that occurs at the ear 108. The hearable 102 can perform this sensing without the use of other auxiliary sensors, such as an optical sensor or an electrical sensor. Through audioplethysmography 110, the hearable 102 can perform biometric monitoring 112, speech detection 114, chewing detection 116, and/or gesture recognition 118. Biometric monitoring 112 can include determining (or measuring) a heart rate, a respiration rate, heart-rate variability, and/or blood pressure of the user 106. Heart rate variability is a shift in timing between heart beats and can reflect a physiological state and/or an emotional state of the user 106. For example, heart rate variability can indicate a heart condition or a mental health issue such as anxiety or depression. With biometric monitoring 112, the user 106 can actively monitor their health and take appropriate action based on any changes in their biometrics to live a longer and healthier life.

Speech detection 114 enables the hearable 102 to determine whether or not the user 106 is speaking. By providing speech detection 114, audioplethysmography 110 can enhance voice control. Voice control enables a user to interact with the computing device 104 in a non-physical and less cognitively demanding way compared to other interfaces that require physical touch and/or the user's visual attention. Additionally or alternatively, speech detection 114 can be used to prevent unauthorized individuals from accessing a voice-control interface of the computing device 104. In particular, audioplethysmography 110 can provide an aspect of multi-factor authentication and confirm that the user 106 is speaking while the computing device 104 recognizes a voiceprint phrase. This can prevent an individual with malicious intent from attempting to access the voice-control interface by playing a recording of the user's voiceprint to the computing device 104. In this manner, speech detection 114 can enhance security of the computing device 104 and provide robust protection from voice attacks.

Chewing detection 116 enables the hearable 102 to monitor for bruxism. Bruxism is a condition in which the user 106 grinds or clenches their teeth, usually in an unconscious manner. In severe cases, bruxism can cause excessive wear on teeth and jaw tenderness, which can result in teeth loss, an abnormal bite, or crooked teeth. It can also be an indication of sleep apnea or stress. With chewing detection 116, the hearable 102 can determine the frequency and duration of teeth griding and communicate this information to the user 106 either directly via the hearable 102 or indirectly via the computing device 104. With this awareness, the user 106 can choose to seek medical advice or make changes to their life-style to reduce the occurrence of bruxism. In some cases, the hearable 102 can sound an alarm to wake the user 106 and cause them to stop grinding their teeth. With this alarm, the user 106 can train themselves to reduce or stop teeth grinding.

Additionally or alternatively, chewing detection 116 can capture a time of day in which the user 106 starts and stops eating. This can be particularly helpful for automatically monitoring intermittent fasting or snacking. The user 106 can later review this information to determine how well they adhered to an intermittent fasting plan or how often they are snacking. In some cases, the hearable 102 can estimate the user 106's calorie intake based on the duration of the chewing activity. The hearable 102 can also be used to train children to properly chew their food before swallowing. For example, the hearable 102 can play audio content for the child as the child chews their food and play a sound once the child chew a target number of times before swallowing.

Gesture recognition 118 enables the hearable 102 to recognize gestures that involve the user 106 engaging different body parts or interacting with different regions on their upper body. A simple ear pull or shake of the head can be detected by the hearable 102 and used to control the computing device 104. More specifically, audioplethysmography 110 can detect subtle pressure waves that originate on the user 106's upper body and propagate to the ear canal. These pressure waves modify characteristics of acoustic signals that are transmitted and received by the hearable 102 and propagate through the ear canal. With gesture recognition 118, the hearable 102 can support a larger quantity and a larger variety of gesture-based controls compared to the limited touch-based controls of some hearables. This is because the user 106 can utilize an entire region of their upper body for different gesture-based controls whereas the touch-based controls are limited to the surface of other hearables. The gesture-based controls can also be subtle so as to not attract attention, particularly during a social event.

To use audioplethysmography 110, the user 106 positions the hearable 102 in a manner that creates at least a partial seal 120 around or in the ear 108. Some parts of the ear 108 are shown in FIG. 1-1, including an ear canal 122 and an ear drum 124 (or tympanic membrane). Due to the seal 120, the hearable 102, the ear canal 122, and the ear drum 124 couple together to form an acoustic circuit. Audioplethysmography 110 involves, at least in part, measuring properties associated with this acoustic circuit. The properties of the acoustic circuit can change due to a variety of different situations or actions.

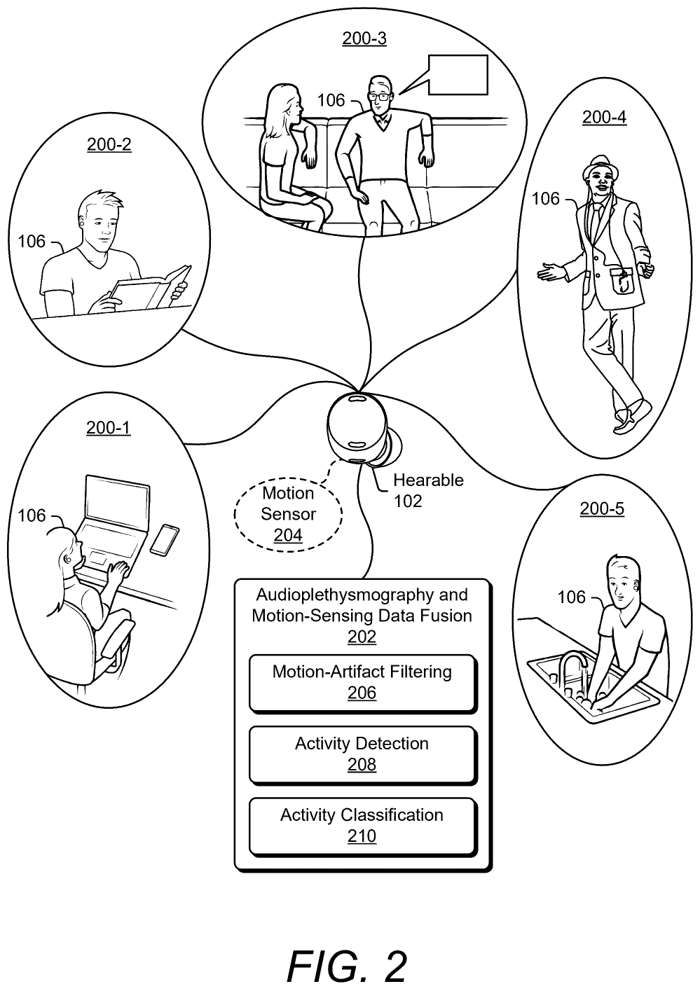

For example, consider FIG. 1-2 in which a change occurs in a physical structure of the ear 108. Example changes to the physical structure include a change in a geometric shape of the ear canal 122 and/or a change in a volume of the ear canal 122. This change can be caused, at least in part, by subtle blood vessel deformations in the ear canal 122 caused by the user 106's heart pumping. Other changes can also be caused by movement in the ear drum 124 or movement of the user 106's jaw.

At 126, for instance, the tissue around the ear canal 122 and the ear drum 124 itself are slightly “squeezed” due to blood vessel deformation. This squeeze causes a volume of the ear canal 122 to be slightly reduced at 126. At 128, however, the squeezing subsides and the volume of the ear canal 122 is slightly increased relative to 126. The physical changes within the ear 108 can modulate an amplitude and/or phase of an acoustic signal that propagates through the ear canal 122, as further described below.

During audioplethysmography 110, an acoustic signal propagates through at least a portion of the ear canal 122. The hearable 102 can receive an acoustic signal that represents a superposition of multiple acoustic signals that propagate along different paths within the ear canal 122. Each path is associated with a delay (τ) and an amplitude (a). The delay and amplitude can vary over time due to the subtle changes that occur in the volume of the ear canal 122. The received acoustic signal can be represented by Equation 1:

$$S ( t ) = n + {\sum }_{i = 1}^{N - 1} {a}_{i} ( t ) cos ( {φ}_{ini} + {Ω}_{fc} ( t + {τ}_{i} ( t ) ) ) \qquad Equation 1$$

where S(t) represents the received acoustic signal, n represents noise, φᵢₙᵢ represents a relative phase between the received acoustic signal and the transmitted acoustic signal, Ω_{fc} represents a frequency of the transmitted acoustic signal, and/represents a time vector. Cardiac activities of the user 106, for instance, can modulate the amplitude and/or phase of the receive acoustic signal, as further shown in Equation 2:

$$S ( t ) = n + ( 1 + {h}_{amp} ( t ) ) cos ( {h}_{phase} ( t ) + {φ}_{ini} ++ {Ω}_{fc} ( t ) ) \qquad Equation 2$$

where hₐₘₚ(t) represents an amplitude modulator and hₚₕₐₛₑ(t) represents a phase modulator. The interactions between the hearable 102 and the ear 108 as well as the physiological activities of the user 106 modulate the amplitude and phase of the received acoustic signal. The techniques for audioplethysmography 110 can be performed while the hearable 102 is playing audible content to the user 106. Various challenges can occur in performing audioplethysmography 110 while the user 106 is actively moving or performing an activity. To address this, the hearable 102 can perform audioplethysmography and motion-sensing data fusion, as further described with respect to FIG. 2.

FIG. 2 illustrates example environments 200-1 to 200-5 in which audioplethysmography and motion-sensing data fusion 202 can be implemented. In the environments 200-1 to 200-5, the user 106 performs a variety of different activities while wearing the hearable 102. The hearable 102 includes at least one motion sensor 204 or has access to data generated by a motion sensor 204 that is implemented separate from the hearable 102, such as implemented within the computing device 104.

In the environments 200-1, 200-2, and 200-3, the user 106 engages in activities that use a relatively low amount of energy. For example, the user 106 works at a desk in the environment 200-1. In the environment 200-2, the user 106 reads a book. In the environment 200-3, the user 106 speaks to another person. Other low-energy activities can include eating, driving, and sleeping. In general, low-energy activities can involve relatively small movements or movements that occur after relatively long intervals of little to no motion. In some cases, the user 106 can be considered relatively motionless while performing the activity. Although the user 106 may be characterized as being relatively motionless, the user 106 may still move slightly due to normal body functions, such as breathing, or due to natural muscle movements.

For some of these low-energy activities, the user 106 may be relatively stationary with respect to coordinates of a Global Navigation Satellite System (GNSS). For example, the user 106 may be sitting, standing, or lying down at a particular location. For other low-energy activities, the user 106 may be relatively stationary within a vehicle (e.g., a car, a train, or an airplane) as the vehicle travels to different locations.

Many low-energy activities can involve the user 106 minimally moving their appendages and/or their body. Sometimes the user 106 may move their head while performing low-energy activities. For example, the user 106 in the environment 200-1 may move their head to take a break from looking at a computer monitor to look out a window. The user 106 in the environment 200-2 may move their head as they scan across pages of a book. Also, the user 106 in the environment 200-3 may move their head as they communicate with the other person.

In the environments 200-4 and 200-5, the user 106 participates in activities that involve a relatively substantial amount of energy compared to the low-energy activities described in the environments 200-1 to 200-3. In the environment 200-4, for instance, the user 106 moves a significant portion of their body. The user 106 can be dancing, as shown in FIG. 2, or performing other activities such as walking, running, biking, skateboarding, roller skating, swimming, exercising, playing a sport, and so forth. In the environment 200-5, the user 106 performs chores, such as washing dishes, cooking, folding laundry, mowing the lawn, gardening, shoveling snow, and so forth.

For some high-energy activities, the user 106 may be physically moving to different coordinates of the Global Navigation Satellite System using their own power. As an example, the user 106 can be biking to a store. For other high-energy activities, the user 106 may be stationary relative to a coordinate of the Global Navigation Satellite System but substantially and/or rapidly moving an appendage or their body. At a gym, for instance, the user 106 can engage in a high-energy activity by exercising on a treadmill or an elliptical machine. Within the environments 200-1 and 200-5, the general motion of the user 106, and more specifically the motion of the user 106's head, can significantly impact audioplethysmography 110.

To address challenges associated with motion and provide additional features for the user 106, the hearable 102 can perform audioplethysmography and motion-sensing data fusion 202. Audioplethysmography and motion-sensing data fusion 202 uses motion-sensing data generated by the motion sensor 204 to process audio signals associated with audioplethysmography 110. Example features that can be implemented as part of audioplethysmography and motion-sensing data fusion 202 include motion-artifact filtering 206, activity detection 208, and/or activity classification 210. Activity classification 210 can also be referred to as activity recognition.

With motion-artifact filtering 206, the hearable 102 can improve sensitivity and accuracy for audioplethysmography 110 while the user is performing a low-energy or a high-energy activity. For example, biometric monitoring 112 can utilize motion-artifact filtering 206 to accurately measure the user 106's heart rate while the user 106 is jogging. Motion-artifact filtering 206 can also be used to reduce a false-alarm rate or false detections associated with other use cases of audioplethysmography 110, such as speech detection 114, chewing detection 116, and/or gesture recognition 118. Other hearables that do not utilize motion-artifact filtering 206 may be unable to accurately collect data using audioplethysmography 110 while the user 106 is moving, which can significantly limit the usefulness of audioplethysmography 110 and present an inconvenience for the user 106.

Activity detection 208 uses audioplethysmography and motion-sensing data fusion 202 to determine that the user 106 is moving. Information about when and how often the user 106 moves can provide additional data for a variety of different use cases. Sleep quality analysis, for instance, can utilize this information to estimate how well the user 106 slept. A similar analysis can be performed to determine meditation frequency and/or quality. Other use cases can include automatically detecting the user 106 taking a break to stand and/or walk around after sitting at a computer and alerting the user 106 if they haven't taken a break within a predetermined amount of time.

Activity classification 210 uses audioplethysmography and motion-sensing data fusion 202 to determine the type of activity the user 106 is performing. While some devices can utilize a motion sensor to perform aspects of activity classification, fusing data provided by the motion sensor 204 with data generated by audioplethysmography 110 can further disambiguate similar activities, which enables a larger quantity of activities to be recognized. In one aspect, data from biometric monitoring 112 through audioplethysmography 110 can be used along with the motion-sensing data to accurately classify an activity performed by the user 106. In another aspect, motion artifacts present within signals associated with audioplethysmography 110 can be used to increase confidence and/or further identify motion detected by the motion sensor 204.

With activity classification 210, the hearable 102 can distinguish between low-energy activities and/or high-energy activities. For example, activity classification 210 can distinguish between the user 106 mixing food by hand or rowing, for instance. In some implementations, activity classification 210 can further distinguish between different types of low-energy activities and high-energy activities. For low-energy activities, the hearable 102 can use activity classification 210 to identify when the user 106 is eating and when the user 106 is talking. For high-energy activities, the hearable 102 can identify when the user 106 is walking and when the user 106 is running. In general, data collected using activity detection 208 and/or activity classification 210 can be used to support a variety of different use cases, including health monitoring, fitness tracking, stress tracking, and so forth.

Activity detection 208 and/or activity classification 210 can also be used to control an operation of the hearable 102 and/or the computing device 104. For example, activity detection 208 can dynamically increase a volume of audio content that is presented by the hearable 102 if activity is detected, or decrease the volume if activity is no longer detected. Activity classification 210 can automatically set a stopwatch, play music that the user previously selected, or open a fitness tracking application of the computing device 104 upon determining that the user 106 is exercising.

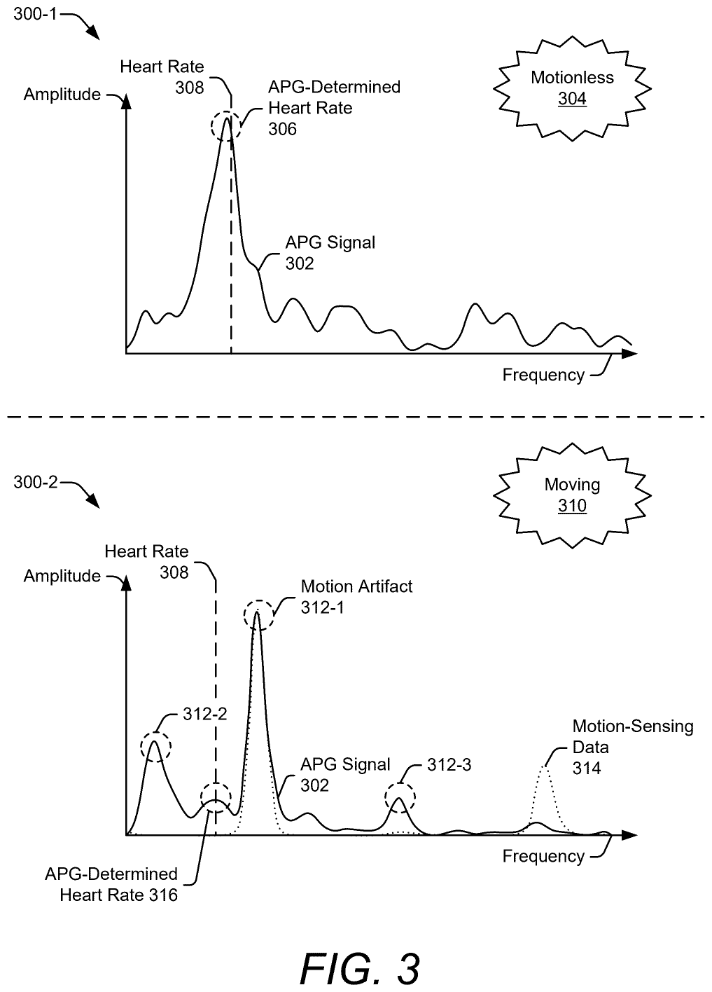

Although motion-artifact filtering 206, activity detection 208, and activity classification 210 can be described with respect to audioplethysmography and motion-sensing data fusion 202, these processes can also generally be associated with audioplethysmography 110. Audioplethysmography 110 involves the transmitting, receiving, and processing of acoustic signals, which can include performing one or more aspects of audioplethysmography and motion-sensing data fusion 202. The challenges associated with performing audioplethysmography 110 while the user 106 moves is further explained with respect to FIG. 3.

FIG. 3 illustrates an impact of motion on audioplethysmography 110. At the top of FIG. 3, a first graph 300-1 depicts amplitude and frequency of an audioplethysmography signal 302 (APG signal 302). In this case, the audioplethysmography signal 302 is collected while the user 106 is relatively motionless 304. The user 106 may be performing a low-energy activity, such as any of the activities described with respect to the environments 200-1 to 200-3. In this case, the user 106 is not moving their head (e.g., their head is relatively stationary). The audioplethysmography signal 302 represents a processed version of a received acoustic signal.

In this example, audioplethysmography 110 is used for biometric monitoring 112. Audioplethysmography 110, for instance, can measure the heart rate of the user 106 based on a highest peak amplitude of the audioplethysmography signal 302. This measured heart rate is referred to as an audioplethysmography-determined heart rate 306 (APG-determined heart rate 306). The audioplethysmography-determined heart rate 306 is approximately equal to the true heart rate 308 of the user 106. The term “approximately” can mean that the audioplethysmography-determined heart rate 306 is within 5% of the heart rate 308 of the user 106 (e.g., within 5%, 3%, 2%, or 1% of the heart rate 308).

As the user 106 is relatively motionless 304 for the example shown in the first graph 300-1, the audioplethysmography signal 302 has relatively little noise and the highest peak amplitude corresponds with the user 106's heart rate 308. As the user 106 moves or participates in high-energy activities, noise caused by the motion of the user 106 can negatively impact the audioplethysmography signal 302. This noise can make it challenging to detect the heart rate 308 of the user 106, as further described below.

At the bottom of FIG. 3, a second graph 300-2 depicts amplitude and frequency of another audioplethysmography signal 302, which is collected while the user 106 is moving 310. In this example, the user 106 is slowly walking. Other examples are also possible in which the user 106 is performing any of the high-energy activities described with respect to the environments 200-4 and 200-5 in FIG. 2.

Similar processing can be used to generate the audioplethysmography signals 302 shown in the graphs 300-1 and 300-2. The audioplethysmography signal 302 in the second graph 300-2, however, includes several motion artifacts 312 that are not present in the first graph 300-1. A first motion artifact 312-1 represents a highest peak amplitude of the audioplethysmography signal 302 and also corresponds with a cadence of the user 106's steps while walking. Other motion artifacts 312-2 and 312-3 are also present in the second graph 300-2. The motion artifacts 312-2 and 312-3 can represent a harmonic or an intermodulation product of the motion artifact 312-1. Additionally or alternatively, the motion artifacts 312-2 and/or 312-3 can be associated with other motions, such as the movement of the user 106's head or arms.

Assuming that audioplethysmography 110 determines the user 106's heart rate based on the highest peak amplitude of the audioplethysmography signal 302, audioplethysmography 110 could incorrectly identify a frequency of the motion artifact 312-1 as the user 106's heart rate. To avoid these inaccuracies, techniques utilizing audioplethysmography and motion-sensing data fusion 202 can attenuate the motion artifacts 312 to enable detection of the desired information (e.g., the heart rate 308). With audioplethysmography and motion-sensing data fusion 202, the audioplethysmography signal 302 is further processed based on motion-sensing data 314, which is also depicted in the second graph 300-2. In particular, motion-artifact filtering 206, which is performed based on the motion-sensing data 314, can be used to filter one or more of the motion artifacts 312-1 to 312-3 and enable audioplethysmography 110 to correctly measure the user 106's heart rate 308. The heart rate 308 measured using audioplethysmography and motion-sensing data fusion 202 is represented by audioplethysmography-determined heart rate 316. The frequencies of the audioplethysmography signals 302 depicted within the graphs 300-1 and 300-2 can be downconverted to baseband frequencies to facilitate alignment with the motion-sensing data 314. The computing device 104 is further described with respect to FIG. 4.

FIG. 4 illustrates an example implementation of the computing device 104. The computing device 104 is illustrated with various non-limiting example devices including a desktop computer 104-1, a tablet 104-2, a laptop 104-3, a television 104-4, a computing watch 104-5, computing glasses 104-6, a gaming system 104-7, a microwave 104-8, and a vehicle 104-9. Other devices may also be used, such as an augmented and/or virtual reality headset, a home service device, a smart speaker, a smart thermostat, a baby monitor, a Wi-Fi™ router, a drone, a trackpad, a drawing pad, a netbook, an e-reader, a home automation and control system, a wall display, and another home appliance. Note that the computing device 104 can be wearable, non-wearable but mobile, or relatively immobile (e.g., desktops and appliances).

The computing device 104 includes one or more computer processors 402 and at least one computer-readable medium 404, which includes memory media and storage media. Applications and/or an operating system (not shown) embodied as computer-readable instructions on the computer-readable medium 404 can be executed by the computer processor 402 to provide some of the functionalities described herein. The computer-readable medium 404 also includes an audioplethysmography-based application 406, which uses information provided by the hearable 102 to perform an action. Example actions can include displaying data associated with audioplethysmography 110 to the user 106. More specifically, the data can be associated with biometric monitoring 112, speech detection 114, chewing detection 116, gesture recognition 118, activity detection 208 and/or activity classification 210.

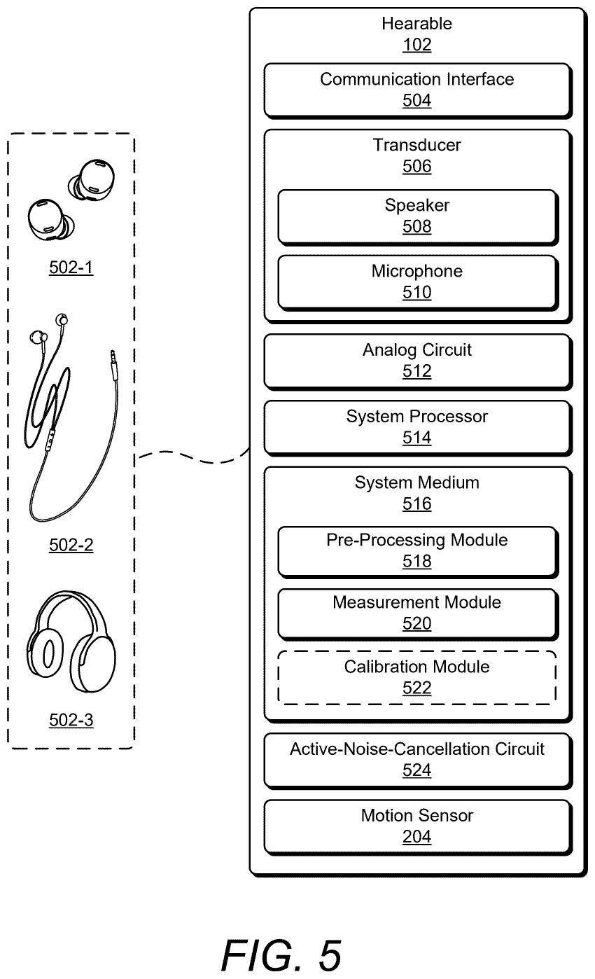

The computing device 104 can also include a network interface 408 for communicating data over wired, wireless, or optical networks. For example, the network interface 408 may communicate data over a local-area-network (LAN), a wireless local-area-network (WLAN), a personal-area-network (PAN), a wire-area-network (WAN), an intranet, the Internet, a peer-to-peer network, point-to-point network, a mesh network, Bluetooth R), and the like. The computing device 104 may also include the display 410. Although not explicitly shown, the hearable 102 can be integrated within the computing device 104, or can connect physically or wirelessly to the computing device 104. The hearable 102 is further described with respect to FIG. 5.

FIG. 5 illustrates an example hearable 102. The hearable 102 is illustrated with various non-limiting example devices, including wireless earbuds 502-1, wired earbuds 502-2, and headphones 502-3. The earbuds 502-1 and 502-2 are a type of in-ear device that fits into the ear canal 122. Each earbud 502-1 or 502-2 can represent a hearable 102. Headphones 502-3 can rest on top of or over the ears 108. The headphones 502-3 can represent closed-back headphones, open-back headphones, on-ear headphones, or over-ear headphones. Each headphone 502-2 includes two hearables 102, which are physically packaged together. In general, there is one hearable 102 for each ear 108.

The hearable 102 includes a communication interface 504 to communicate with the computing device 104, though this need not be used when the hearable 102 is integrated within the computing device 104. The communication interface 504 can be a wired interface or a wireless interface, in which audio content is passed from the computing device 104 to the hearable 102. The hearable 102 can also use the communication interface 504 to pass data associated with audioplethysmography 110 to the computing device 104. In general, the data provided by the communication interface 504 is in a format usable by the audioplethysmography-based application 406.

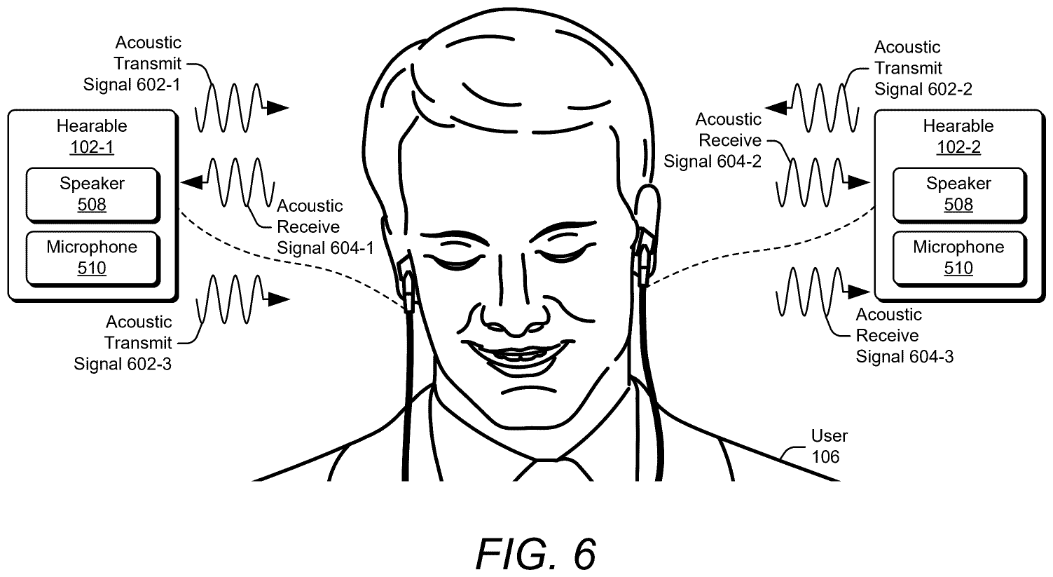

The communication interface 504 also enables the hearable 102 to communicate with another hearable 102. During bistatic sensing, for instance, the hearable 102 can use the communication interface 504 to coordinate with the other hearable 102 to support two-ear audioplethysmography 110, as further described with respect to FIG. 6. In particular, the transmitting hearable 102 can communicate timing and waveform information to the receiving hearable 102 to enable the receiving hearable 102 to appropriately demodulate a received acoustic signal.

The hearable 102 includes at least one transducer 506 that can convert electrical signals into sound waves. The transducer 506 can also detect and convert sound waves into electrical signals. These sound waves may include ultrasonic frequencies and/or audible frequencies, either of which may be used for audioplethysmography 110. In particular, a frequency spectrum (e.g., range of frequencies) that the transducer 506 uses to generate an acoustic signal can include frequencies from a low-end of the audible range to a high-end of the ultrasonic range, e.g., between 20 hertz (Hz) to 2 megahertz (MHZ). Other example frequency spectrums for audioplethysmography 110 can encompass frequencies between 20 Hz and 20 kilohertz (kHz), between 20 KHz and 2 MHZ, between 20 and 96 kHz, between 20 and 60 kHz, or between 30 and 40 KHz.

In an example implementation, the transducer 506 has a monostatic topology. With this topology, the transducer 506 can convert the electrical signals into sound waves and convert sound waves into electrical signals (e.g., can transmit or receive acoustic signals). Example monostatic transducers may include piezoelectric transducers, capacitive transducers, and micro-machined ultrasonic transducers (MUTs) that use microelectromechanical systems (MEMS) technology.

Alternatively, the transducer 506 can be implemented with a bistatic topology, which includes multiple transducers that are physically separate. In this case, a first transducer converts the electrical signal into sound waves (e.g., transmits acoustic signals), and a second transducer converts sound waves into an electrical signal (e.g., receives the acoustic signals). An example bistatic topology can be implemented using at least one speaker 508 and at least one microphone 510. The speaker 508 and the microphone 510 can be dedicated for audioplethysmography 110 or can be used for both audioplethysmography 110 and other functions of the computing device 104 (e.g., presenting audible content to the user 106, capturing the user 106's voice for a phone call, or for voice control).

In general, the speaker 508 and the microphone 510 are directed towards the ear canal 122 (e.g., oriented towards the ear canal 122). Accordingly, the speaker 508 can direct acoustic signals towards the ear canal 122, and the microphone 510 is responsive to receiving acoustic signals from the direction associated with the ear canal 122.

The hearable 102 includes at least one analog circuit 512, which includes circuitry and logic for conditioning electrical signals in an analog domain. The analog circuit 512 can include analog-to-digital converters, digital-to-analog converters, amplifiers, filters, mixers, and switches for generating and modifying electrical signals. In some implementations, the analog circuit 512 includes other hardware circuitry associated with the speaker 508 or microphone 510.

The hearable 102 also includes at least one system processor 514 and at least one system medium 516 (e.g., one or more computer-readable storage media). In the depicted configuration, the system medium 516 includes a pre-processing module 518 and a measurement module 520. The system medium 516 also optionally includes a calibration module 522. The pre-processing module 518, the measurement module 520, and the calibration module 522 can be implemented using hardware, software, firmware, or a combination thereof. In this example, the system processor 514 implements the pre-processing module 518, the measurement module 520, and the calibration module 522. In an alternative example, the computer processor 402 of the computing device 104 can implement at least a portion of the pre-processing module 518, the measurement module 520, and the calibration module 522. In this case, the hearable 102 can communicate digital samples of the acoustic signals to the computing device 104 using the communication interface 504.

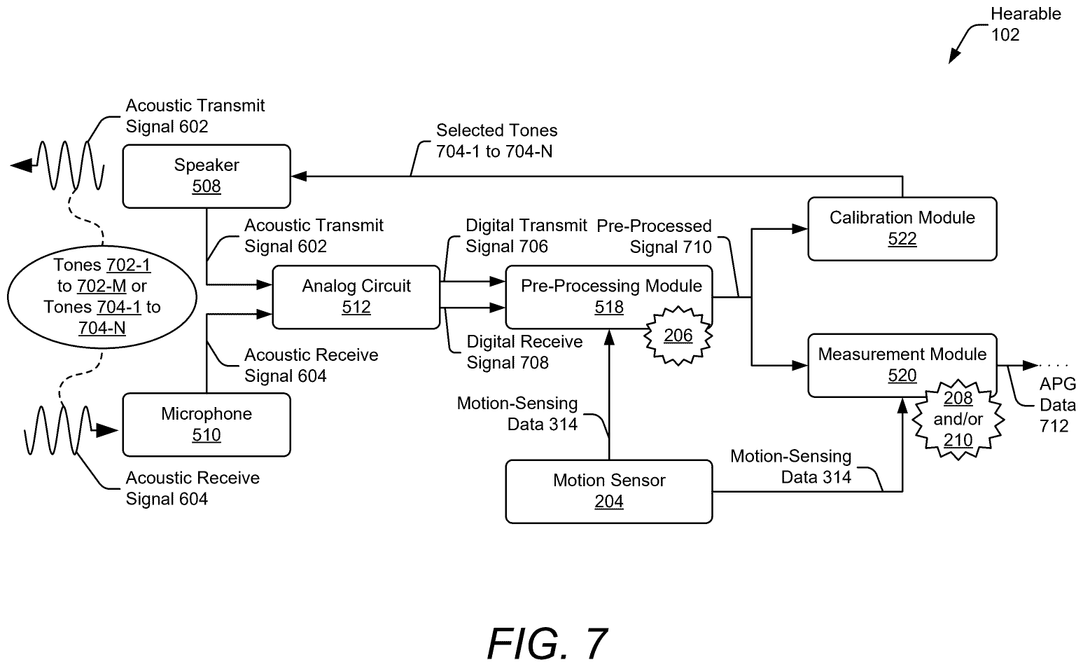

Operations of the pre-processing module 518, the measurement module 520, and the calibration module 522 are further described with respect to FIGS. 7-10. Aspects of audioplethysmography and motion-sensing data fusion 202 can be performed by the pre-processing module 518 and/or the measurement module 520, as further described with respect to FIGS. 11-1 to 12.

Some hearables 102 include an active-noise-cancellation circuit 524, which enables the hearables 102 to reduce background or environmental noise. In this case, the microphone 510 used for audioplethysmography 110 can be implemented using a feedback microphone of the active-noise-cancellation circuit 524. During active noise cancellation, the feedback microphone provides feedback information regarding the performance of the active noise cancellation. During audioplethysmography 110, the feedback microphone receives an acoustic signal, which is provided to the pre-processing module 518. In some situations, active noise cancellation and audioplethysmography 110 are performed simultaneously using the feedback microphone. In this case, the acoustic signal received by the feedback microphone can be provided to the pre-processing module 518 and the active-noise-cancellation circuit 524.

The hearable 102 can also include at least one motion sensor 204. Example motion sensors 204 include an inertial measurement unit (IMU), an accelerometer, an inclinometer, a gyroscope, a magnetometer, a Global Navigation Satellite System, or some combination thereof. In general, the motion sensor 204 can detect and/or measure one or more characteristics of motion. Some motion sensors 204, for instance, can measure linear acceleration and/or a rotational velocity (or angular velocity), detect changes in orientation, detect changes in inclination, or some combination thereof. The linear accelerations and the rotational velocities can be associated with one, two, or three orthogonal axes. The motion sensor 204 generates the motion-sensing data 314 for audioplethysmography and motion-sensing data fusion 202. The motion-sensing data 314 can include time-series data associated with the measured linear accelerations and/or rotational velocities. Other types of motion-sensing data 314 can include indications of changes in orientation and/or inclination, coordinates measured by the Global Navigation Satellite System, and so forth. The motion-sensing data 314 can include one or more of the characteristics of motion described above.

Other implementations are also possible in which the motion sensor 204 is separate from the hearable 102. For example, the motion sensor 204 can be implemented within the computing device 104. As another example, the motion sensor 204 can be a separate device that is physically attached to the user 106 and communicatively coupled to the hearable 102 and/or the computing device 104. In this case, audioplethysmography and motion-sensing data fusion 202 can be designed to account for differences between the motion-sensing data 314 provided by the motion sensor 204 and the motion observed by the hearable 102 using audioplethysmography 110. Different types of audioplethysmography 110 are further described with respect to FIG. 6.

### Audioplethysmography

FIG. 6 illustrates example operations of two hearables 102-1 and 102-2. In a first example operation, the hearables 102-1 and 102-2 perform single-ear audioplethysmography 110. This means that the hearables 102-1 and 102-2 independently perform audioplethysmography 110 on different ears 108 of the user 106. In this case, the first hearable 102-1 is proximate to the user 106's right ear 108, and the second hearable 102-2 is proximate to the user 106's left ear 108. Each hearable 102-1 and 102-2 includes a speaker 508 and a microphone 510. The hearables 102-1 and 102-2 can operate in a monostatic manner during the same time period or during different time periods. In other words, each hearable 102-1 and 102-2 can independently transmit and receive acoustic signals.

For example, the first hearable 102-1 uses the speaker 508 to transmit a first acoustic transmit 602-1, which propagates within at least a portion of the user 106's right ear canal 122. The first hearable 102-1 uses the microphone 510 to receive a first acoustic receive signal 604-1. The first acoustic receive signal 604-1 represents a version of the first acoustic transmit signal 602-1 that is modified, at least in part, by the acoustic circuit associated with the right ear canal 122. This modification can change an amplitude, phase, and/or frequency of the first acoustic receive signal 604-1 relative to the first acoustic transmit signal 602-1.

Similarly, the second hearable 102-2 uses the speaker 508 to transmit a second acoustic transmit signal 602-2, which propagates within at least a portion of the user 106's left ear canal 122. The second hearable 102-2 uses the microphone 510 to receive a second acoustic receive signal 604-2. The second acoustic receive signal 604-2 represents a version of the second acoustic transmit signal 602-2 that is modified by the acoustic circuit associated with the left ear canal 122. This modification can change an amplitude, phase, and/or frequency of the second acoustic receive signal 604-2 relative to the second acoustic transmit signal 602-2.

The techniques of single-ear audioplethysmography 110 can be particularly beneficial as it enables the computing device 104 to compile information from both hearables 102-1 and 102-2, which can further improve measurement confidence. For some aspects of audioplethysmography 110, it can be beneficial to analyze the acoustic channel between two ears 108, as further described below.

In a second example operation, the two hearables 102-1 and 102-2 perform two-ear audioplethysmography 110. This means that the hearables 102-1 and 102-2 jointly perform audioplethysmography 110 across two ears 108 of the user 106. In this case, at least one of the hearables 102 (e.g., the first hearable 102-1) includes the speaker 508, and at least one of the other hearables 102 (e.g., the second hearable 102-2) includes the microphone 510. The hearables 102-1 and 102-2 operate together in a bistatic manner during the same time period.

During operation, the first hearable 102-1 transmits a third acoustic transmit 402-3 using the speaker 508. The third acoustic transmit signal 602-3 propagates through the user 106's right ear canal 122. The third acoustic transmit signal 602-3 also propagates through an acoustic channel that exists between the right and left ears 108. In the left ear 108, the third acoustic transmit signal 602-3 propagates through the user 106's left ear canal 122 and is represented as a third acoustic receive signal 604-3. The second hearable 102-2 receives the third acoustic receive signal 604-3 using the microphone 510. The third acoustic receive signal 604-3 represents a version of the third acoustic transmit signal 602-3 that is modified by the acoustic circuit associated with the right ear canal 122, modified by the acoustic channel associated with the user 106's face, and modified by the acoustic circuit associated with the left ear canal 122. This modification can change an amplitude, phase, and/or frequency of the third acoustic receive signal 604-3 relative to the third acoustic transmit signal 602-3. In some cases, the hearable 102-2 measures the time-of-flight (ToF) associated with the propagation from the first hearable 102-1 to the second hearable 102-2. Sometimes a combination of single-ear and two-ear audioplethysmography 110 are applied to further improve measurement confidence.

The acoustic transmit signals 602 of FIG. 6 can represent a variety of different types of signals. As described above with respect to FIG. 5, the acoustic transmit signal 602 can be an ultrasonic signal and/or an audible signal. Also, the acoustic transmit signal 602 can be a continuous-wave signal (e.g., a sinusoidal signal) or a pulsed signal. Some acoustic transmit signals 602 can have a particular tone (or frequency). Other acoustic transmit signals 602 can have multiple tones (or multiple frequencies). A variety of modulations can be applied to generate the acoustic transmit signal 602. Example modulations include linear frequency modulations, triangular frequency modulations, stepped frequency modulations, phase modulations, or amplitude modulations. The acoustic transmit signal 602 can be transmitted as part of a calibration procedure or a measurement procedure, as further described as part of FIG. 7.

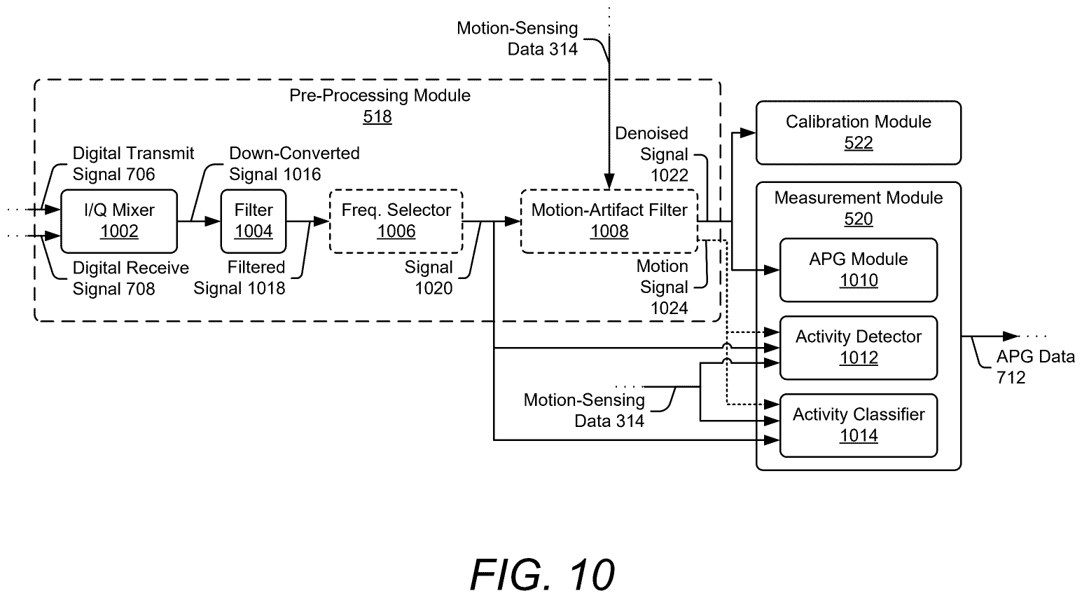

FIG. 7 illustrates an example implementation of the hearable 102 for performing audioplethysmography and motion-sensing data fusion 202. In the depicted configuration, the hearable 102 includes the speaker 508, the microphone 510, the analog circuit 512, the pre-processing module 518, the measurement module 520, the calibration module 522, and the motion sensor 204. Other implementations of the hearable 102, however, are also possible in which the hearable 102 does not include the calibration module 522 to reduce processing power requirements. In this case, the pre-processing module 518 can perform aspects of frequency selection as further described with respect to FIG. 10 to improve the signal-to-noise ratio for audioplethysmography 110. Some hearables 102 may not include the motion sensor 204 to save cost and/or to reduce a footprint of the hearable 102. In this case, the motion sensor 204 can be communicatively coupled to the hearable 102 and implemented in another manner. For example, the motion sensor 204 can be implemented within the computing device 104 or implemented as a separate entity that is physically attached to the user 106.

Outputs of the speaker 508 and the microphone 510 are coupled to inputs of the analog circuit 512. The pre-processing module 518 has inputs that are coupled to outputs of the analog circuit 512 and an output of the motion sensor 204. The pre-processing module 518 also has outputs that are coupled to inputs of the measurement module 520 and the calibration module 522. The measurement module 520 has another input that is coupled to the motion sensor 204. The calibration module 522 has an output that is coupled to the speaker 508.

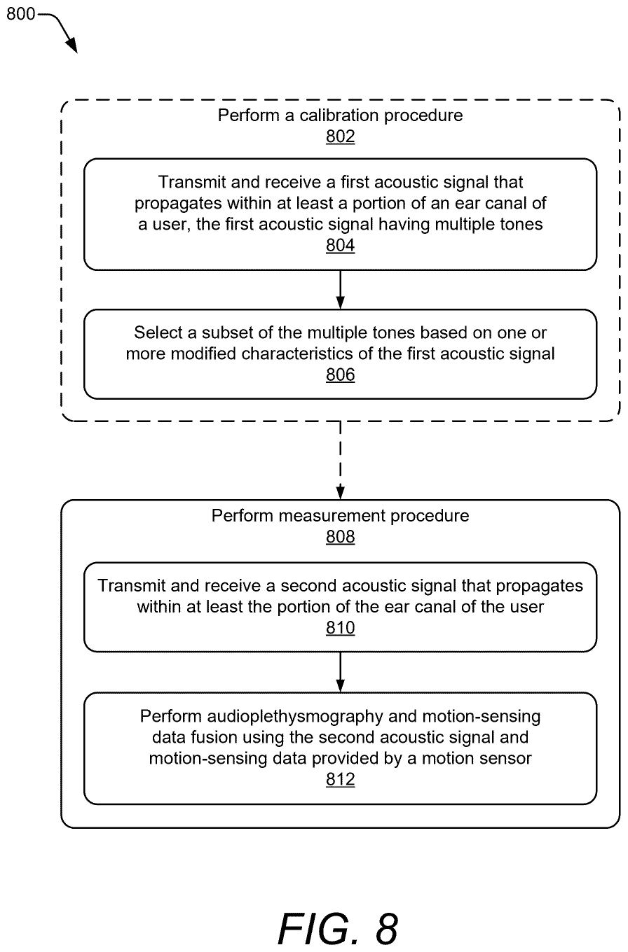

Consider an example operation of the hearable 102 in accordance with single-ear audioplethysmography 110. In the case that the hearable 102 includes the calibration module 522, the hearable 102 can perform a calibration process prior to performing a measurement process. The calibration process and the measurement process are further described with respect to FIG. 8.

During both the calibration process and the measurement process, the speaker 508 transmits the acoustic transmit signal 602, and the microphone 510 receives the acoustic receive signal 604. During the calibration process, the acoustic transmit signal 602 and the acoustic receive signal 604 can have tones 702-1 to 702-M, where M represents a positive integer. During the measurement process, the acoustic transmit signal 602 and the acoustic receive signal 604 can have selected tones 704-1 to 704-N, where N represents a positive integer that is less than or equal to M. The selected tones 704-1 to 704-N can represent a subset (sometimes a proper subset) of the tones 702-1 to 702-M.

The analog circuit 512 performs analog-to-digital conversion to generate a digital transmit signal 706 and a digital receive signal 708 based on the acoustic transmit signal 602 and the acoustic receive signal 604, respectively. The pre-processing module 518 performs frequency downconversion and demodulation to generate at least one pre-processed signal 710 based on the digital transmit signal 706 and the digital receive signal 708. The pre-processing module 518 can also apply filtering to generate the pre-processed signal 710. For the calibration procedure and/or the measurement procedure, the pre-processing module 518 can optionally apply motion-artifact filtering 206 to generate the pre-processed signal 710 based on the digital receive signal 708 and the motion-sensing data 314.

As part of the calibration procedure, the calibration module 522 processes the pre-processed signal 710 to determine the selected tones 704-1 to 704-N. The selected tones 710-1 to 710-N can improve performance of audioplethysmography 110 during the measurement procedure. The calibration module 522 communicates the selected tones 704-1 to 704-N to the speaker 508 using a control signal. The speaker 508 accepts the control signal that identifies the selected tones 704-1 to 710-N and can transmit a subsequent acoustic transmit signal 602 for the measurement procedure using the selected tones 704-1 to 704-N.

As part of the measurement procedure, the measurement module 520 can perform aspects of audioplethysmography 110 using the pre-processed signal 710 to generate audioplethysmography data 712 (APG data 712). The audioplethysmography data 712 can be communicated to the audioplethysmography-based application 406. Additionally or alternatively, the measurement module 520 can perform audioplethysmography and motion-sensing data fusion 202 using the pre-processed signal 710 and the motion-sensing data 314. In particular, the measurement module 520 can perform activity detection 208 and/or activity classification 210. With activity detection 208 and/or activity classification 210, the audioplethysmography data 712 can include an indication of whether or not an activity is detected and/or the type of activity that is detected. Additionally or alternatively, the audioplethysmography data 712 can include a control signal for controlling an operation of the hearable 102 and/or the computing device 104. The calibration procedure and the measurement procedure are further described with respect to FIG. 8.

FIG. 8 illustrates an example flow diagram 800 for operating a hearable 102. In FIG. 8, the hearable 102 can optionally perform a calibration procedure at 802 using the calibration module 522. The calibration procedure can determine appropriate characteristics (e.g., waveform or signal characteristics) of transmitted acoustic signals 602 to improve audioplethysmography 110 (e.g., to enhance the performance of audioplethysmography 110). The calibration procedure enables audioplethysmography 110 to take into account the wear of the hearable 102 (e.g., the position of the hearable 102 relative to the ear canal 122) and the physical structure of the ear canal 122 to determine a transmission frequency that can increase accuracy performance. As an example, the use of the calibration procedure can enable the hearable 102 to determine a biometric of the user 106 with an accuracy of 95% or more. With the calibration procedure, the hearable 102 can dynamically adjust the transmission frequency (e.g., one or more carrier frequencies) each time the seal 120 is formed (e.g., based on the wear of the hearable 102) and based on the unique physical structure of the ear 108. Through this calibration procedure, the hearables 102 on different ears 108 may operate with one or more different acoustic frequencies. Steps of the calibration procedure are further described below:

In some circumstances, the hearable 102 can perform on-head detection (or in-ear detection) by detecting the presence of the seal 120 and initiating the calibration procedure based on a determination that on-head detection is “true.” In other circumstances, the hearable 102 can initiate the calibration procedure based on a specified schedule or a timer, which can be controlled by the user 106 via the computing device 104.

At 804, the hearable 102 executes the calibration procedure by transmitting and receiving a first acoustic signal. The first acoustic signal propagates within at least a portion of the ear canal 122 of the user 106 and has multiple tones 702-1 to 702-M (or multiple carrier frequencies). The multiple tones 702-1 to 702-M are transmitted in parallel or in series over a given time interval. The first acoustic transmit signal 602 can have a particular bandwidth on the order of several kilohertz. For example, the acoustic transmit signal 602 can have a bandwidth of approximately 4, 5, 6, 8, 10, 16, or 20 KHz. In example implementations, the first acoustic transmit signal 602 is transmitted over multiple seconds, such as 2, 3, 4, 6, or more seconds. A duration of each tone 702 can be evenly divided over a total duration of the first acoustic transmit signal 602.

In an example implementation, the acoustic transmit signal 602 has seven tones 702 (e.g., M equals 7). In some cases, the tones 702 are evenly distributed across an interval. For example, the tones 702 can be in 1 kHz increments between 32 kHz and 38 kHz (e.g., at approximately 32, 33, 34, 35, 36, 37, and 38 kHz). The term “approximately” means that the tones 702 can be within 5% of a given value or less (e.g., within 3%, 2%, or 1% of the given value).

An amplitude of the acoustic transmit signal 602 can be approximately the same across the tones 702-1 to 702-M. In this manner, power is evenly distributed across each tone 702. The quantity of tones 702 (e.g., M) can be determined based on an output power of the speaker 508. Increasing the quantity of tones 702 can increase a likelihood that the hearable 102 can support a given use case across various conditions including user wear and a physical structure of the user 106's ear canal 122. However, an amplitude of the acoustic transmit signal 602 can be limited across these tones 702 based on the output power of the speaker 508. Thus, the quantity of tones 702 can be optimized based on an amount of output power that is available for audioplethysmography 110.

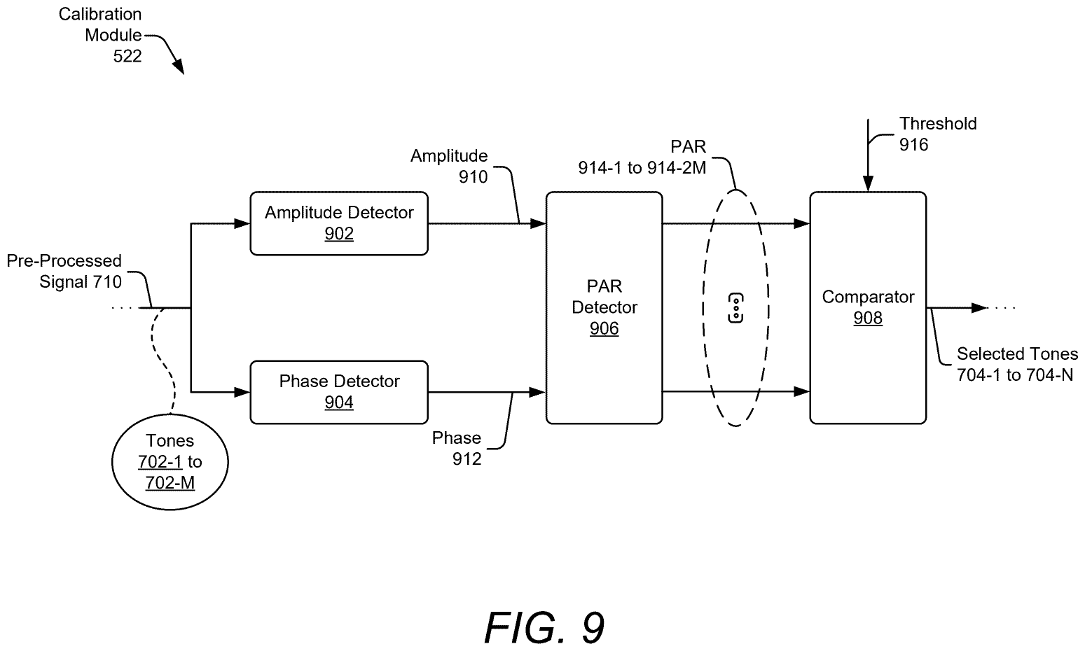

At 806, the calibration procedure selects one or more tones 704-1 to 704-N to be used for a measurement procedure based on one or more modified characteristics of the acoustic receive signal 604. The process for selecting the tones 704 is further described with respect to FIG. 9. In general, the calibration procedure determines that the selected tones 704 improve a signal-to-noise ratio for audioplethysmography 110.

At 808, the hearable 102 performs a measurement procedure using the measurement module 520. In accordance with the measurement procedure, the hearable 102 transmits a second acoustic transmit signal 602 that propagates within at least the portion of the ear canal 122 of the user 106. If the calibration procedure was performed, the second acoustic transmit signal 602 can have the selected tones 704-1 to 704-N that were determined by the calibration procedure. The selected tones 704 can be transmitted in parallel or in series over a given time interval.

An amplitude of the second acoustic transmit signal 602 can be approximately the same across the selected tones 704-1 to 704-N. In this manner, power is evenly distributed across each selected tone. The amplitude of the second acoustic transmit signal 602 can be higher than the amplitude of the first acoustic transmit signal 602 because the available output power is distributed across fewer tones. Additionally or alternatively, a duration of each of the selected tones 704 of the second acoustic transmit signal 602 can be longer than the duration of the tones 702 of the first acoustic transmit signal 602. The higher amplitude and/or the longer duration can further improve the signal-to-noise ratio performance of the hearable 102 for audioplethysmography 110. By using a few selected tones 704 that were determined to improve signal-to-noise ratio performance, the measurement procedure can achieve a higher accuracy for audioplethysmography 110.

At 812, the hearable 102 performs audioplethysmography and motion-sensing data fusion 202 using the second acoustic signal (e.g., the second acoustic receive signal 604) and the motion-sensing data 314 provided by the motion sensor 204. This can include performing motion-artifact filtering 206, activity detection 208, and/or activity classification 210, as further described with respect to FIG. 10. The calibration module 522 is further described with respect to FIG. 9.

FIG. 9 illustrates an example scheme implemented by the calibration module 522. In the depicted configuration, the calibration module 522 implements a frequency selector, which selects one or more tones 704 for the measurement procedure. In the example implementation, the calibration module 522 includes at least one amplitude detector 902, at least one phase detector 904, at least one peak-to-average ratio (PAR) detector 906 (PAR detector 906), and at least one comparator 908. The operations of these components are further described below.

During the calibration procedure, the calibration module 522 accepts the pre-processed signal 710 from the pre-processing module 518, as previously described with respect to FIG. 7. The pre-processed signal 710 can include amplitude and/or phase information associated with the multiple tones 702-1 to 702-M, which were used to transmit the first acoustic signal described at 802 in FIG. 8.

In this example, the calibration module 522 extracts an amplitude 910 of the pre-processed signal 710 using the amplitude detector 902 and extracts a phase 912 of the pre-processed signal 710 using the phase detector 904. Alternatively, if in-phase and quadrature components of the pre-processed signal 710 are received separately, the amplitude detector 902 and the phase detector 904 can respectively measure the amplitude 910 and phase 912 based on the in-phase and quadrature components.

The peak-to-average ratio detector 906 measures peak-to-average ratios 914-1 to 914-2M for each of the tones 702-1 to 702-M and for each of the characteristics (e.g., amplitude 910 and phase 912). In general, the peak-to-average ratio 914 represents a peak intensity within a frequency range of interest divided by an average intensity within this frequency range. For biometric monitoring 112, the frequency range can be, for example, between 0.58 and 3.3 Hz to correspond with a possible range of a human heart rate, which can be between 35 and 200 beats-per-minute. Other frequency ranges can be used for other use cases, including for measuring other biometrics, speech detection 114, chewing detection 116, gesture recognition 118, activity detection 208, and/or activity classification 210. A higher peak-to-average ratio 914 indicates a higher-quality signal, or more generally, higher signal-to-noise ratio performance.

In one aspect, the comparator 908 can evaluate the peak-to-average ratios 914-1 to 914-2M with respect to a threshold 916. The threshold 916 can be set, for example, to a particular value, such as four. In other cases, the calibration module 522 can dynamically determine the threshold 916 and update it over time based on the observed peak-to-average ratios 914-1 of 914-2M. In an example implementation, the comparator 908 determines the selected tones 704-1 to 704-N for a subsequent measurement procedure based on the frequencies associated with the peak-to-average ratios 914-1 to 914-2M that are greater than or equal to the threshold 916.

Additionally or alternatively, the comparator 908 can evaluate the peak-to-average ratios 914-1 to 914-2M with respect to each other. In an example implementation, the comparator 908 determines one of the selected tones 704 based on a frequency with the highest peak-to-average ratio 914 across the amplitude 910. Also, the comparator 908 can determine one of the selected tones 704 based on a frequency with the highest peak-to-average ratio 914 across the phase 912. In other implementations, the comparator 908 can determine a single selected tone 704 based on a frequency having the highest peak-to-average ratio 914 associated with either the amplitude 910 or the phase 912.

In general, the calibration module 522 enables the selected tones 704-1 to 704-N to be dynamically adjusted prior to the measurement procedure based on a current environment, which can account for a wear of the hearable 102 (e.g., a current insertion depth and/or rotation), a physical structure of the user 106's ear canal 122, and a response characteristic of the hearable 102 (e.g., speaker, microphone, and/or housing). In this manner, the calibration module 522 can improve the signal-to-noise ratio performance of the hearable 102 for the measurement procedure. The calibration module 522 can also determine which tones 704 generate acoustic receive signals 604 with desired characteristics for a given use case. For biometric monitoring 112, for instance, the calibration module 522 can identify which tones 704 generate detectable cardiac modulations in amplitude 910 and/or phase 912.

In FIGS. 7 to 9, the calibration procedure and the measurement procedure are described as individual procedures that occur at different time intervals. In particular, the calibration procedure occurs before the measurement procedure. This enables the acoustic transmit signal 602 for the measurement procedure to be transmitted with fewer tones than the acoustic transmit signal 602 used for the calibration procedure, which can increase signal-to-noise ratio performance for audioplethysmography 110. In some implementations, however, the hearable 102 can have sufficient output power to perform the measurement procedure with the multiple tones 702-1 to 702-M using a single acoustic transmit signal 602. In this case, aspects of the calibration module can be integrated within the pre-processing module 518 as a frequency selector, as further described with respect to FIG. 10. This frequency selector can effectively pass the selected tones 704-1 to 704-N for further processing. Aspects of the measurement procedure are further described with respect to FIG. 10.

### Audioplethysmography and Motion-Sensing Data Fusion

FIG. 10 illustrates an example scheme implemented by the hearable 102 for performing audioplethysmography and motion-sensing data fusion 202. In the depicted configuration, the hearable 102 includes the pre-processing module 518, which is coupled to the measurement module 520 and the calibration module 522. The pre-processing module 518 and/or the measurement module 520 are also coupled to the motion sensor 204 (not shown).

The pre-processing module 518 includes at least one in-phase and quadrature mixer 1002 (I/Q mixer 1002) and at least one filter 1004. The in-phase and quadrature mixer 1002 performs frequency down-conversion. In an example implementation, the in-phase and quadrature mixer 1002 includes at least two mixers, at least one phase shifter, and at least one combiner (e.g., a summation circuit). The filter 1004 attenuates intermodulation products that are generated by the in-phase and quadrature mixer 1002. In an example implementation, the filter 1004 is implemented using a low-pass filter.

The pre-processing module 518 can optionally include at least one frequency selector 1006 and/or at least one motion-artifact filter 1008. The motion-artifact filter 1008 can perform motion-artifact filtering 206 to improve performance of audioplethysmography 110. Example implementations of the motion-artifact filter 1008 are further described with respect to FIGS. 11-1 and 11-2.

The frequency selector 1006 can identify and select one or more tones 704 (or carrier frequencies) that provide a high-quality signal for later processing. The frequency selector 1006 can further pass the selected tones 704 to other processing modules and filter (or attenuate) other tones that are not selected. The frequency selector 1006 can be implemented in a similar manner as the calibration module 522 of FIG. 9. For example, the frequency selector 1006, can include the amplitude detector 902, the phase detector 904, the peak-to-average ratio detector 906, and the comparator 908.

The measurement module 520 can include at least one audioplethysmography module 1010 (APG module 1010), at least one activity detector 1012, at least one activity classifier 1014, or some combination thereof. The audioplethysmography module 1010 processes information provided by the pre-processing module 518 to generate audioplethysmography data 712 for any of the described use cases, including biometric monitoring 112, speech detection 114, chewing detection 116, and/or gesture recognition 118.

For measuring the heart-rate variability as part of biometric monitoring 112, for instance, the audioplethysmography module 1010 can use peak finding estimation to localize a peak of each heartbeat within the pre-processed signal 710. This estimation can be performed across the amplitude and/or phase of the pre-processed signal 710. Example peak finding estimation techniques include Z-score, local maxima, and divide and conquer. The audioplethysmography module 1010 can measure the variability of the heart rate by calculating a root mean square of successive differences (RMSSD) between each peak (e.g., between each heartbeat).

In some cases, the audioplethysmography module 1010 can provide the audioplethysmography data 712 to other components of the measurement module 520, such as the activity detector 1012 or the activity classifier 1014. In this way, the audioplethysmography data 712 can be used to improve activity detection 208 and/or activity classification 210. Consider an example implementation in which the audioplethysmography module 1010 performs chewing detection 116 or gesture recognition 118 and determines that the user 106 is chewing or performing a gesture. This information, as part of the audioplethysmography data 712, can be used by the activity detector 1012 in addition to the motion-sensing data 314 to determine that the user 106 is moving. In another example implementation, the audioplethysmography module 1010 performs biometric monitoring 112 and measures the user 106's heart rate and/or respiration rate. This information, as part of the audioplethysmography data 712, can be used by the activity classifier 1014 in addition to the motion-sensing data 314 to recognize an activity that is being performed by the user 106.

In general, the activity detector 1012 and/or the activity classifier 1014 process information provided by the pre-processing module 518 (or the audioplethysmography module 1010) and the motion sensor 204 to generate the audioplethysmography data 712. In particular, the activity detector 1012 performs activity detection 208, and the activity classifier 1014 performs activity classification 210.

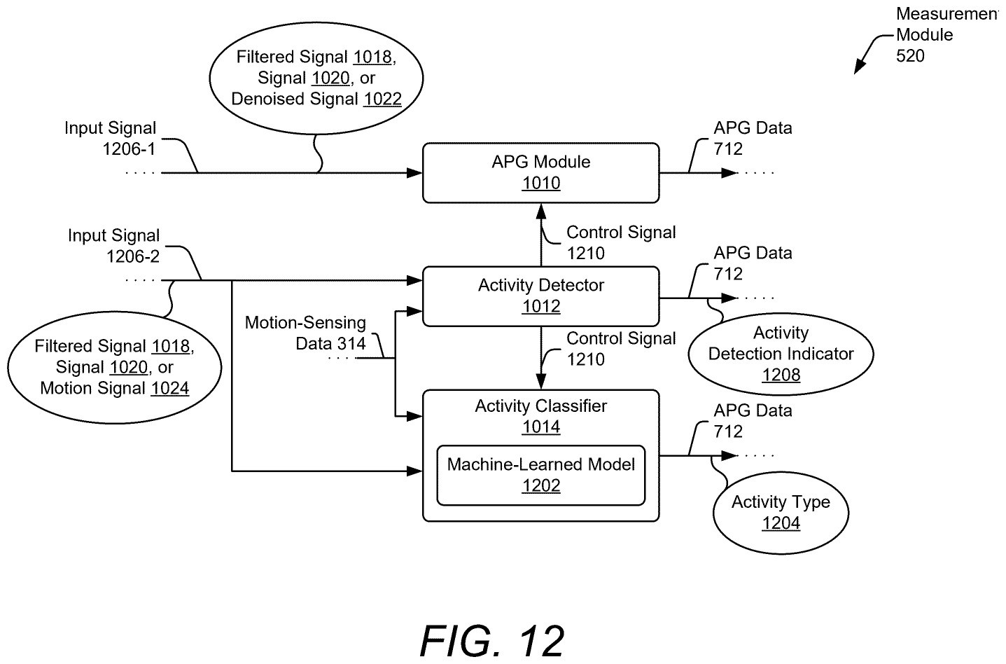

In some cases, the audioplethysmography module 1010 can operate independently of the activity detector 1012 and/or the activity classifier 1014. In other cases, the activity detector 1012 and/or the activity classifier 1014 can pass information to the audioplethysmography module 1010, which can modify an operation of the audioplethysmography module 1010 or modify the audioplethysmography data 712 that is generated by the audioplethysmography module 1010. In still other cases, the audioplethysmography module 1010 can pass information to the activity detector 1012 and/or the activity classifier 1014 to enhance activity detection 208 and/or activity classification 210. Example implementations of the activity detector 1012 and the activity classifier 1014 are further described with respect to FIG. 12.

During an operation, the in-phase and quadrature mixer 1002 uses the phase shifter and the two mixers to generate in-phase and quadrature components associated with the digital receive signal 708. In particular, the in-phase and quadrature mixer 1002 mixes the digital receive signal 708 with a first version of the digital transmit signal 706 that has a zero-degree phase shift to generate the in-phase component. Additionally, the in-phase and quadrature mixer 1002 mixes the digital receive signal 708 with a second version of the digital transmit signal 706 that has a 180-degree phase shift to generate the quadrature signal. This mixing operation downconverts the digital receive signal 708 from acoustic frequencies to baseband frequencies. Using the combiner, the in-phase and quadrature mixer 1002 combines the in-phase and quadrature components of the digital receive signal 708 to generate a down-converted signal 1016. Use of the in-phase and quadrature mixer 1002 can further improve the signal-to-noise ratio of the down-converted signal 1016 compared to other mixing techniques.

In this example, the down-converted signal 1016 represents a combination of the in-phase and quadrature components of the mixed-down digital receive signal 708. In alternative implementations, the in-phase and quadrature mixer 1002 doesn't include the combiner and passes the in-phase and quadrature components separately to the filter 1004. In this manner, the in-phase and quadrature components individually propagate through the filter 1004.

The filter 1004 generates a filtered signal 1018 based on the down-converted signal 1016. In particular, the filter 1004 filters the down-converted signal 1016 to attenuate spurious or undesired frequencies (e.g., intermodulation products), some of which can be associated with an operation of the in-phase and quadrature mixer 1002. In this example, the filtered signal 1018 represents a combination of the in-phase and quadrature components of the down-converted signal 1016. Alternatively, the filtered signal 1018 can represent separate or distinct in-phase and quadrature components, which are individually passed to the frequency selector 1006 and/or the motion-artifact filter 1008.

During the measurement procedure, the pre-processing module 518 can optionally apply the frequency selector 1006. The frequency selector 1006 passes tones that meet a quality threshold level of performance for audioplethysmography 110. For example, the frequency selector 1006 pass tones 704 having an amplitude 910 and/or phase 912 with a peak-to-average ratio 914 that is greater than or equal to a threshold 916. The resulting signal outputted by the frequency selector 1006 is represented by signal 1020. In some implementations, this signal 1020 is passed to the measurement module 520 and/or the calibration module 522 as the pre-processed signal 710. In other implementations in which the frequency selector 1006 is not implemented, the filtered signal 1018 can be passed to the measurement module 520 and/or the calibration module 522 as the pre-processed signal 710.

The pre-processing module 518 can optionally apply the motion-artifact filter 1008 during the measurement procedure and/or the calibration procedure. The motion-artifact filter 1008 generates a denoised signal 1022 based on an input signal and the motion-sensing data 314. The input signal can be the filtered signal 1018 or the signal 1020.

Generally speaking, the motion-artifact filter 1008 uses the motion-sensing data 314 to remove and/or significantly attenuate motion artifacts 312 that are present in the input signal. Within the denoised signal 1022, this attenuation can cause the motion artifact 312 to have an amplitude that is less than an amplitude of a desired signal component that is associated with the propagation of the acoustic signal within the ear canal 122 of the user 106. Compared to the input signal, the denoised signal 1022 has fewer motion artifacts 312 and/or motion artifacts 312 with significantly smaller amplitudes.

In some implementations, the motion-artifact filter 1008 can also generate a motion signal 1024. The motion signal 1024 includes the motion artifacts 312 of the input signal. The motion signal 1024 does not include signal components associated with the propagation within the ear canal 122.

The audioplethysmography module 1010 can generate the audioplethysmography data 712 based on the denoised signal 1022. By using the denoised signal 1022 to perform audioplethysmography 110 instead of the filtered signal 1018 or the signal 1020, the audioplethysmography module 1010 can improve accuracy and reduce false detections in situations in which the user 106 is moving or performing an activity (e.g., a low-energy activity and/or a high-energy activity).

The activity detector 1012 can perform activity detection 208 and/or the activity classifier 1014 can perform activity classification 210 based on the pre-processed signal 710 provided by the pre-processing module 518 and the motion-sensing data 314. The pre-processed signal 710 can represent the filtered signal 1018, the signal 1020 (as shown in FIG. 10), or the motion signal 1024 (as shown in FIG. 10 using dashed lines). Use of the activity detector 1012 and/or the activity classifier 1014 can provide additional features for the hearable 102, as further described with respect to FIG. 12. Example implementations of the motion-artifact filter 1008 are further described with respect to FIGS. 11-1 and 11-2.

### Motion-Artifact Filtering

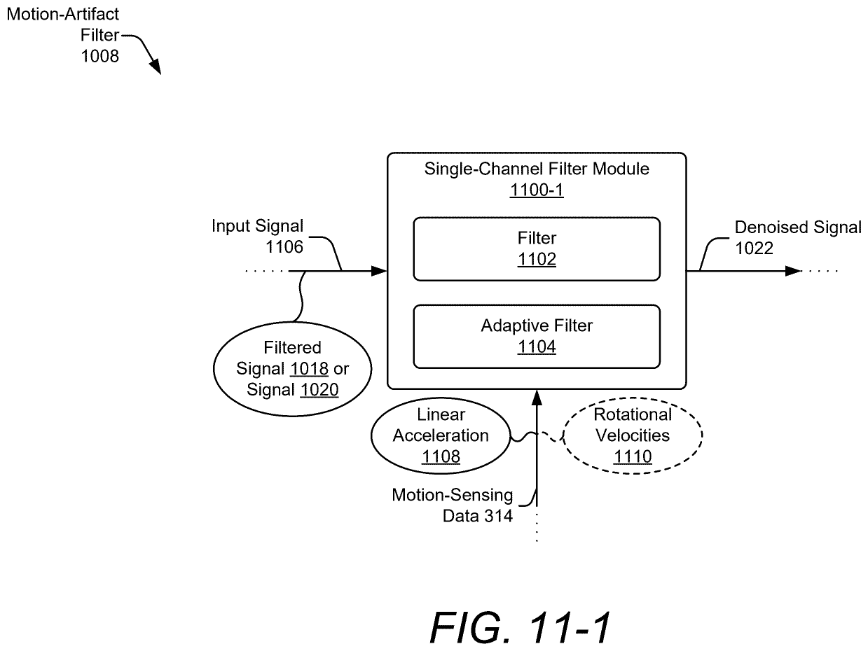

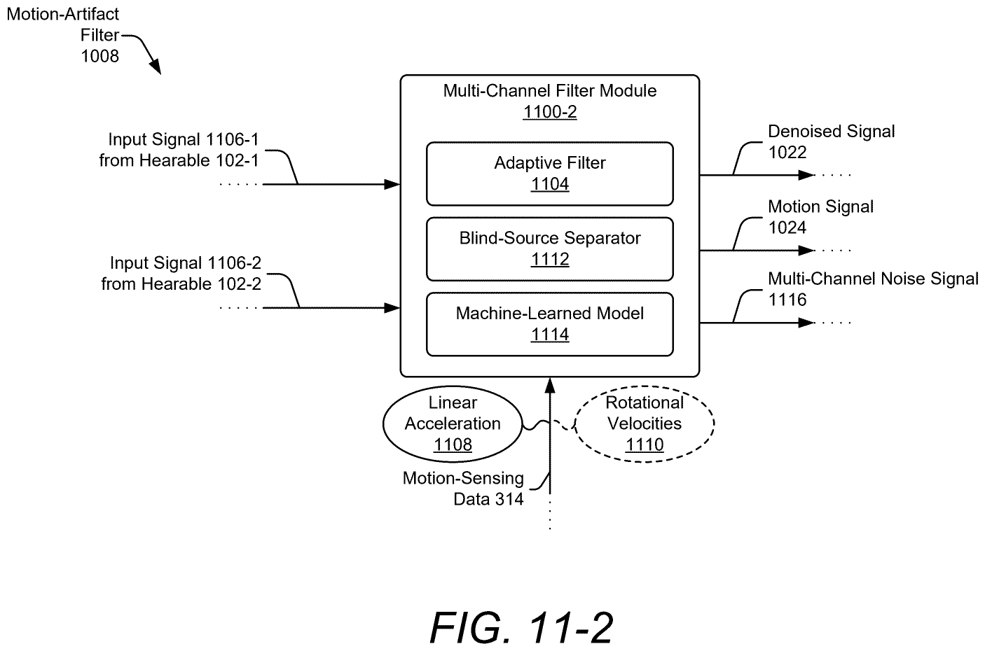

FIG. 11-1 illustrates a first example implementation of the motion-artifact filter 1008 for performing aspects of audioplethysmography and motion-sensing data fusion 202. In the depicted configuration, the motion-artifact filter 1008 includes at least one single-channel filter module 1100-1, which performs motion artifact filtering 206. The single-channel filter module 1100-1 can be implemented using at least one filter 1102 or at least one adaptive filter 1104. During operation, the single-channel filter module 1100-1 filters an input signal 1106 based on the motion-sensing data 314 to generate the denoised signal 1022. The input signal 1106 can include the filtered signal 1018 or the signal 1020. The motion-sensing data 314 at least includes linear accelerations 1108 for three axes. Optionally, the motion-sensing data 314 can include rotational velocities 1110. Other implementations are also possible in which other types of motion-sensing data 314 is used in addition to or instead of the linear accelerations 1108.

In a first example implementation, the single-channel filter module 1100-1 uses the filter 1102 to generate the denoised signal 1022. The filter 1102 is capable of performing informed filtering based on the motion-sensing data 314. With informed filtering, the filter 1102 attenuates motion artifacts within the input signal 1106 that are also present in the motion-sensing data 314. The motion artifacts 312 within the input signal 1106 can have frequencies that are related to frequencies associated with motion artifacts 312 within the motion-sensing data 314. In general, these frequencies are based on the frequencies associated with motion (e.g., cadence of a user's stride while walking). By referencing the motion-sensing data 314, the filter 1102 can significantly attenuate the frequencies associated with the motion artifacts 312 within the input signal 1106 and pass (or minimally attenuate) other frequencies within the input signal 1106 that are not associated with the motion artifacts 312. Explained another way, the filter 1102 represents a conventional filter, such as a low-pass filter, a high-pass filter, a band-pass filter, or some combination thereof, having at least one cutoff frequency that can be dynamically tuned (or dynamically adjusted) based on the frequencies observed within the motion-sensing data 314. In this case, the frequencies of interest for audioplethysmography 110 differ from the frequencies associated with possible motion artifacts 312. In this manner, the frequencies of interest are not attenuated by the filter 1102.

In a second example implementation, the single-channel filter module 1100-1 uses the adaptive filter 1104 to generate the denoised signal 1022. With adaptive filtering techniques, the input signal 1106 represents a primary reference and the motion-sensing data 314 represents a noise reference. The adaptive filter 1104 uses the motion-sensing data 314 to remove motion artifacts 312 within the input signal 1106. Various adaptive filtering techniques can be used, including techniques based on least mean squares (LMS) or recursive least squares (RLS).

The single-channel filter module 1100-1 can operate with a single-channel input signal, which in this case represents a filtered signal 1018 or a signal 1020 that is associated with the hearable 102-1 or the hearable 102-2. Other multi-channel implementations of the motion-artifact filter 1008 are also possible, as further described with respect to FIG. 11-2.

FIG. 11-2 illustrates a second example implementation of the motion-artifact filter 1008 for performing aspects of audioplethysmography and motion-sensing data fusion 202. In the depicted configuration, the motion-artifact filter 1008 includes at least one multi-channel filter module 1100-2, which performs motion artifact filtering 206. The multi-channel filter module 1100-2 can be implemented using the adaptive filter 1104, at least one blind-source separator 1112 or at least one machine-learned model 1114.

During operation, the multi-channel filter module 1100-2 accepts input signals 1106-1 and 1106-2 respectively associated with the hearables 102-1 and 102-2. The input signals 1106-1 and 1106-2 can each represent a filtered signal 1018 or a signal 1020. The multi-channel filter module 1100-2 generates the denoised signal 1022, the motion signal 1024, and optionally a multi-channel noise signal 1116 based on the input signals 1106-1 and 1106-2 and the motion-sensing data 314. The motion-sensing data 314 at least includes linear accelerations 1108 for three axes. Optionally, the motion-sensing data 314 can include rotational velocities 1110. Other implementations are also possible in which other types of motion-sensing data 314 is used in addition to or instead of the linear accelerations 1108.

The denoised signal 1022 can represent a single channel of signal components associated with audioplethysmography 110. For example, the denoised signal 1022 can include signal components that are present within the input signal 1006-1 or the input signal 1006-2. The motion signal 1024 also represents a single channel of motion artifacts 312. For example, the motion signal 1024 can include the motion artifacts 312 that are present within the input signal 1006-1 or the input signal 1006-2. The denoised signal 1022 and the motion signal 1024 can be associated with a same channel (e.g., associated with a same hearable 102-1 or 102-2). The multi-channel noise signal 1116 can include other noise that is not associated with the motion artifacts 312. In contrast to the denoised signal 1022 and the motion signal 1024, the multi-channel noise signal 1116 can be associated with both of the input signals 1106-1 and 1106-2.

The blind-source separator 1112 applies blind source separation (BSS) techniques to separate the motion artifacts 312 from the signal components corresponding to audioplethysmography 110. The blind-source separator 1112 can use various transformation techniques, such as principal component analysis (PCA) with singular value decomposition (SVD).

The machine-learned model 1114 is implemented using one or more neural networks. A neural network includes a group of connected nodes (e.g., neurons or perceptrons), which are organized into one or more layers. As an example, the machine-learned model 1114 includes a deep neural network, which includes an input layer, an output layer, and one or more hidden layers positioned between the input layer and the output layers. The nodes of the deep neural network can be partially-connected or fully-connected between the layers.

In some implementations, the neural network is a recurrent neural network (e.g., a long short-term memory (LSTM) neural network) with connections between nodes forming a cycle to retain information from a previous portion of an input data sequence for a subsequent portion of the input data sequence. In other cases, the neural network is a feed-forward neural network in which the connections between the nodes do not form a cycle. Additionally or alternatively, the machine-learned model 1114 includes another type of neural network, such as a convolutional neural network. The machine-learned model 1114 can also include one or more types of regression models, such as a single linear regression model, multiple linear regression models, logistic regression models, step-wise regression models, multi-variate adaptive regression splines, locally estimated scatterplot smoothing models, and so forth.

In general, the machine-learned model 1114 is trained using supervised learning to generate the denoised signal 1022, the motion signal 1024, and the multi-channel noise signal 1116 based on the input signals 1106-1 and 1106-2. In this way, the machine-learned model 1114 is trained to separate out the motion artifacts 312 and the signal components from the input signals 1106-1 and 1106-2. In some implementations, the machine-learned model 1114 can be further trained to perform one or more aspects of the audioplethysmography module 1010. In general, the supervised learning can use simulated (e.g., synthetic) data or measured (e.g., real) data for training purposes.

With the adaptive filter 1104, the blind-source separator 1112, and/or the machine-learned model 1114, the pre-processing module 518 may not include the frequency selector 1006, as this aspect can be handled by the motion-artifact filter 1008. The measurement module 520 is further described with respect to FIG. 12.

### Activity Detection and or Classification

FIG. 12 illustrates an example implementation of the measurement module 520 for performing aspects of audioplethysmography and motion-sensing data fusion 202. In the depicted configuration, the measurement module 520 includes the audioplethysmography module 1010, the activity detector 1012, and the activity classifier 1014. The activity detector 1012 is coupled to the audioplethysmography module 1010 and the activity classifier 1014.

The activity classifier 1014 is implemented using at least one machine-learned model 1202, which includes one or more neural networks (e.g., a deep neural network). Nodes of a deep neural network can be partially-connected or fully-connected between the layers. In some implementations, the neural network is a recurrent neural network (e.g., a long short-term memory (LSTM) neural network) with connections between nodes forming a cycle to retain information from a previous portion of an input data sequence for a subsequent portion of the input data sequence. In other cases, the neural network is a feed-forward neural network in which the connections between the nodes do not form a cycle. Additionally or alternatively, the machine-learned model 1202 includes another type of neural network, such as a convolutional neural network.

The activity classifier 1014 can also include one or more types of classification models, such as a binary classification model, a multi-class classification model, multi-label classification, and so forth. In general, the machine-learned model 1202 is trained using supervised learning to identify at least one activity type 1204 based on the pre-processed signal 710 and the motion-sensing data 314.

The activity detector 1012 can be implemented using at least one filter, a comparison module, and/or a machine-learned module. In general, the activity detector 1012 can perform aspects of filtering, comparison, correlation, and or classification to determine whether or not the user 106 is moving (e.g., to determine whether or not the user 106 is performing an activity). The machine-learned model used to implement the activity detector 1012 can be similar to the machine-learned model 1202. For example, the machine-learned model can be implemented using a classification model, which classifies input signals as including or not including artifacts associated with motion.

In some implementations, the activity detector 1012 accepts a signal associated with audioplethysmography 110 (e.g., input signal 1206-2) and accepts the motion-sensing data 314. This can enable the activity detector 1012 to further determine is audioplethysmography 110 is impacted by the motion of the user 106, which can be advantageous for situations in which the motion sensor 204 is separate from the hearable 102. In other implementations, the activity detector 1012 can perform activity detection using only the motion-sensing data 314, which can simplify the processing for implementations in which the motion sensor 204 is integrated within the hearable 102.

During operation, the measurement module 520 accepts the pre-processed signal 710 from the pre-processing module 518. In FIG. 12, the pre-processed signal 710 is represented as a first input signal 1206-1 that is provided to the audioplethysmography module 1010 and a second pre-processed signal 1206-2 that is provided to the activity detector 1012 and/or the activity classifier 1014. In various implementations, the input signals 1206-1 and 1206-2 can be the same signal or different signals, as further explained below: In general, the input signal 1206-1 at least includes signal components associated with propagation of an acoustic signal within the ear canal 122 of the user 106. This enables the input signal 1206-1 to be processed for any of the use cases associated with audioplethysmography 110. In contrast, the input signal 1206-2 at least includes one or more motion artifacts 312 associated with motion of the user 106. This enables the input signal 1206-2 to be processed for activity detection 208 and/or activity classification 210.

In some implementations, the input signals 1206-1 and 1206-2 represent a same signal, which can include the filtered signal 1018 or the signal 1020. In other implementations, the input signals 1206-1 and 1206-2 can represent different signals. For example, the first input signal 1206-1 can represent the denoised signal 1022 for implementations in which the pre-processing module 518 includes the motion-artifact filter 1008. In this case, the second pre-processed signal 710-2 can represent the filtered signal 1018 or the signal 1020. For some implementations of the motion-artifact filter 1008, such as implementations that include the multi-channel filter module 1100-2 of FIG. 11-2, the second pre-processed signal 710-2 can represent the motion signal 1024.

As part of the operation of the measurement module 520, the audioplethysmography module 1010 can generate the audioplethysmography data 712 (or a portion thereof) based on the input signal 1206-1. The audioplethysmography data 712 can include information that is associated with one or more characteristics of the acoustic receive signal 604 that are passed to the audioplethysmography module 1010 as part of the input signal 1206-1. In example implementations, the audioplethysmography data 712 can include information that supports biometric monitoring 112, speech detection 114, chewing detection 116, and/or gesture recognition 118.

Additionally or alternatively, the activity detector 1012 can generate the audioplethysmography data 712 (or a portion thereof) based on the input signal 1206-2. The audioplethysmography data 712 can include information that is associated with one or more motion artifacts 312 that are present within the input signal 1206-2 and/or the motion-sensing data 314. In example implementations, the audioplethysmography data 712 generated by the activity detector 1012 can include an activity detection indicator 1208, which identifies whether or not the user 106 moved or engaged in an activity. The activity detection indicator 1208 can be further used to control an operation of the hearable 102 and/or the computing device 104.

In some implementations, the activity detector 1012 generates a control signal 1210, which can be provided to the audioplethysmography module 1010 and/or the activity classifier 1014. The control signal 1210 can dynamically enable and/or disable the audioplethysmography module 1010 and the activity classifier 1014 based on the activity detection indicator 1208.

Consider an example in which the pre-processing module 518 does not perform motion-artifact filtering 206. In this case, the audioplethysmography module 1010 can operate on the filtered signal 1018 and/or the signal 1020, which can include motion artifacts 312 that can negatively impact performance for audioplethysmography 110. To enhance performance for audioplethysmography 110, the control signal 1210 can disable the audioplethysmography module 1010 if the activity detection indicator 1208 indicates that activity (or more generally motion) is detected. In this case, the activity detection indicator 1208 can be communicated to the computing device 104, which can cause the computing device 104 to inform the user 106 that audioplethysmography 110 is currently unavailable. The computing device 104 can also prompt the user 106 to reduce movement so as to enable audioplethysmography 110. Also, the control signal 1210 can enable the audioplethysmography module 1010 if the activity detection indicator 1208 indicates that activity is not detected. In this way, the activity detector 1012 controls when audioplethysmography 110 is performed based on the input signal 1206-2 and/or the motion-sensing data 314.

Consider another example in which power and/or computational resources of the hearable 102 are limited. To conserve power and/or computational resources, the control signal 1210 can disable the activity classifier 1014 if the activity detection indicator 1208 indicates that activity (or more generally motion) is not detected. Also, the control signal 1210 can enable the activity classifier 1014 if the activity detection indicator 1208 indicates that activity is detected. In some cases, the control signal 1210 can enable the activity classifier 1014 after the activity detection indicator 1208 indicates that activity has been occurring for a predetermined time period.

As another part of the operation of the measurement module 520, the activity classifier 1014 can generate the audioplethysmography data 712 (or a portion thereof) based on the input signal 1206-2 and the motion-sensing data 314. The audioplethysmography data 712 can include information that is associated with one or more motion artifacts 312 that are present within the input signal 1206-2 and the motion-sensing data 314. In example implementations, the audioplethysmography data 712 generated by the activity classifier 1014 can include an activity type 1204. In some implementations, the activity type 1204 can indicate whether the activity performed by the user 106 is associated with a low-energy activity or a high-energy activity. In other implementations, the activity type 1204 can further identify the type of low-energy activity and/or the type of high-energy activity. For example, the activity type 1204 can indicate whether the user 106 is speaking, reading, walking, or running.

The activity detection indicator 1208 and/or the activity type 1204 can control an operation of the hearable 102 and/or the computing device 104. For example, either can appropriately adjust a volume of the hearable 102, cause different audio content to be presented to the user 106, control an operation of the audioplethysmography-based application 406, and so forth.

Although not explicitly shown, an output of the activity classifier 1014 can be coupled to an input of the audioplethysmography module 1010 in some implementations. In this case, the audioplethysmography module 1010 can use the activity type 1204 determined by the activity classifier 1014 to improve measurement accuracy and/or customize operation. For example, if the activity type 1204 indicates that the user 106 is performing a high-energy activity and/or exercising, the audioplethysmography module 1010 can switch from performing gesture recognition 118 to performing biometric monitoring 112.

Additionally or alternatively, the audioplethysmography module 1010 can customize the measurement of biometrics based on the activity type 1204 to simplify processing and/or improve accuracy. If the activity type 1204 indicates that the user is exercising, for instance, the audioplethysmography module 1010 can tune a filter to detect the user 106's heart rate and/or respiration rate within a higher range of frequencies. Alternatively, if the activity type 1204 indicates that the user 106 is performing a low-energy activity, the audioplethysmography module 1010 can tune a filter to detect the user 106's heart rate and/or respiration rate within a lower range of frequencies.

### Example Methods

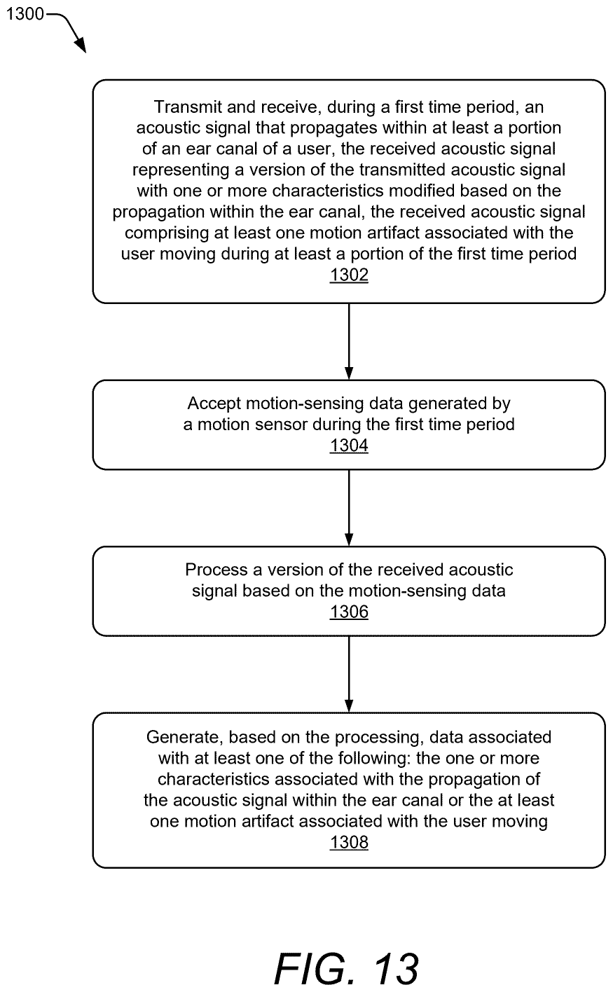

FIGS. 13, 14, and 15 depict example methods 1300, 1400, and 1500 for implementing aspects of audioplethysmography and motion-sensing data fusion 202. Methods 1300, 1400, and 1500 are shown as sets of operations (or acts) performed but not necessarily limited to the order or combinations in which the operations are shown herein. Further, any of one or more of the operations may be repeated, combined, reorganized, or linked to provide a wide array of additional and/or alternate methods. In portions of the following discussion, reference may be made to the environments 200-1 to 200-5 of FIG. 2, and entities detailed in FIGS. 4 and 5, reference to which is made for example only. The techniques are not limited to performance by one entity or multiple entities operating on one device.

At 1302 in FIG. 13, an acoustic signal that propagates within at least a portion of an ear canal of a user is transmitted and received during a first time period. The received acoustic signal represents a version of the transmitted acoustic signal with one or more characteristics modified based on the propagation within the ear canal. The received acoustic signal comprises at least one motion artifact associated with the user moving during at least a portion of the first time period.

For example, the hearable 102 transmits and receives, during a first time period, an acoustic signal that propagates within at least a portion of the ear canal 122 of the user 106, as shown in FIG. 6. Explained another way, the hearable 102 transmits the acoustic transmit signal 602 and receives the acoustic receive signal 604 during the first time period. The transmission and reception can be performed in a half-duplex manner or a full-duplex manner depending on the implementation. In some cases, single-ear audioplethysmography 110 can be used to transmit and receive the acoustic signal using at least one of the hearables 102-1 or 102-2 of FIG. 6. In other cases, two-ear audioplethysmography 110 can be used to transmit the acoustic signal using the hearable 102-1 and receive the acoustic signal using the hearable 102-2.

The acoustic signal can be referred to as the acoustic transmit signal 602 or the acoustic receive signal 604 depending on the context. The received acoustic signal (e.g., the acoustic receive signal 604) represents a version of the transmitted acoustic signal (e.g., the acoustic transmit signal 602) with one or more characteristics modified based on the propagation within the ear canal 122. The one or more modified characteristics can include amplitude, phase, and/or frequency. The received acoustic signal also includes at least one motion artifact 312 associated with the user 106 moving during at least a portion of the first time period. The motion artifact 312 can represent a portion of the received acoustic signal where an amplitude, phase, and/or frequency is based on the user 106's movement.

The version of the received acoustic signal can represent a digital version of the acoustic receive signal 604, a version of the acoustic receive signal 604 that is downconverted to a particular frequency range for further processing (e.g., downconverted to baseband frequencies), a pre-processed version of the acoustic receive signal 604, or some combination thereof. In general, the term “the version of the received acoustic signal” means there can be some differences between the acoustic signal that is received and the acoustic receive signal 604 that is provided for the audioplethysmography-and-motion-sensing-data-fusion process. Explained another way, the version of the acoustic receive signal 604 is related to the acoustic receive signal 604, but can be further modified to support an operation of the hearable 102. In this case, the operation involves audioplethysmography and motion-sensing data fusion 202.

In general, the movement of the user 106 during the first time period corresponds with the user 106 at least moving their head. The motion artifact 312 can be associated with a movement of the user 106's head, such as a translation movement, a rotational movement, a tilting movement, and/or a change in orientation. Example head movements can include the head moving side-to-side, up-and-down, tilting, and so forth. The motion artifact 312 can also be associated with other motions performed by the user 106 as they engage in a particular activity, such as a low-energy activity described with respect to environments 200-1 to 200-3 or a high-energy activity described with respect to environments 200-4 and 200-5. For example, the motion artifact 312 (or another motion artifact 312) can be associated with the movement of the user 106's arms and/or legs.

At 1304, motion-sensing data generated by a motion sensor during the first time period is accepted. For example, the motion sensor 204 generates motion-sensing data 314 during the first time period. The pre-processing module 518 and/or the measurement module 520 accepts (e.g., receives) the motion-sensing data 314. This enables the pre-processing module 518 and/or the measurement module 520 to perform aspects of audioplethysmography and motion-sensing data fusion 202.

At 1306, a version of the received acoustic signal is processed based on the motion-sensing data. For example, the hearable 102 performs audioplethysmography and motion-sensing data fusion 202 by processing the version of the received acoustic signal based on the motion-sensing data 314. More specifically, the hearable 102 can perform motion-artifact filtering 206, activity detection 208, and/or activity classification 210, as shown in FIGS. 2 and 10.

At 1308, data is generated based on the processing. The data is associated with at least one of the following: the one or more characteristics associated with the propagation of the acoustic signal within the ear canal or the at least one motion artifact associated with the user moving. For example, the hearable 102 generates audioplethysmography data 712 based on the processing, as shown in FIG. 7. If at least a portion of the audioplethysmography data 712 is generated by the audioplethysmography module 1010, the audioplethysmography data 712 can be associated with the one or more characteristics of the acoustic receive signal 604 that are associated with the propagation of the acoustic transmit signal 602 within the ear canal 122. More specifically, the audioplethysmography data 712 can include information associated with biometric monitoring 112, speech detection 114, chewing detection 116, and/or gesture recognition 118. Additionally or alternatively, if at least a portion of the audioplethysmography data 712 is generated by the activity detector 1012 or the activity classifier 1014, the audioplethysmography data 712 can be associated with the at least one motion artifact 312.

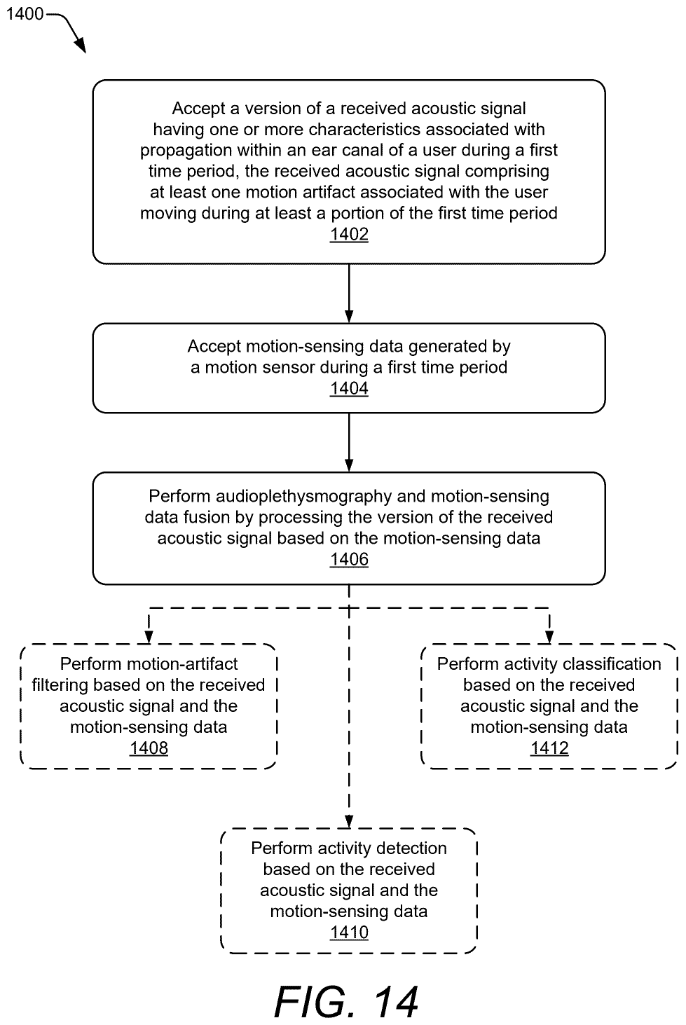

At 1402 in FIG. 14, a version of a received acoustic signal having one or more characteristics associated with propagation within an ear canal of a user during a first time period is accepted. The received acoustic signal comprises at least one motion artifact associated with the user moving during at least a portion of the first time period. For example, the pre-processing module 518 or the measurement module 520 accepts a version of the acoustic receive signal 604 having one or more characteristics associated with propagation within the ear canal of the user 106 during a first time period. The received acoustic signal includes at least one motion artifact 312 associated with the user 106 moving during at least a portion of the first time period.

At 1404, motion-sensing data generated by a motion sensor during a first time period is accepted. For example, the pre-processing module 518 or the measurement module 520 accept the motion-sensing data 314 generated by the motion sensor 204 during the first time period, as described above with respect to 1304 in FIG. 13.

At 1406, audioplethysmography and motion-sensing data fusion is performed by processing the version of the received acoustic signal based on the motion-sensing data. For example, the hearable 102 performs audioplethysmography and motion-sensing data fusion 202 by processing the version of the received acoustic signal 604 based on the motion-sensing data 314. One or more of the steps described at 1408, 1410, and 1412 can be executed to perform an aspect of audioplethysmography and motion-sensing data fusion 202.

Optionally at 1408, motion-artifact filtering is performed based on the received acoustic signal and the motion-sensing data. For example, the motion-artifact filter 1008 performs motion-artifact filtering 206 based on an input signal 1106 and the motion-sensing data 314, as shown in FIG. 10. Various implementations of the motion-artifact filter 1008 can process an input signal 1106 associated with a single channel (or a single hearable 102), as described with respect to FIG. 11-1, or process multiple input signals 1106-1 and 1106-2 associated with multiple channels (or multiple hearables 102-1 and 102-2), as described with respect to FIG. 11-2.

Optionally at 1410, activity detection based on the received acoustic signal and the motion-sensing data is performed. For example, the measurement module 520 performs activity detection 208 based on a pre-processed signal 710 and the motion-sensing data 314, as shown in FIGS. 10 and 12.

Optionally at 1412, activity classification is performed based on the received acoustic signal and the motion-sensing data. For example, the measurement module 520 performs activity classification 210 based on a pre-processed signal 710 and the motion-sensing data 314, as shown in FIGS. 10 and 12.

In some situations, the methods 1300 and/or 1400 are performed using one hearable 102 for single-ear audioplethysmography 110. In other situations, the methods 1300 and/or 1400 are performed using two hearables 102 for two-ear audioplethysmography 110.

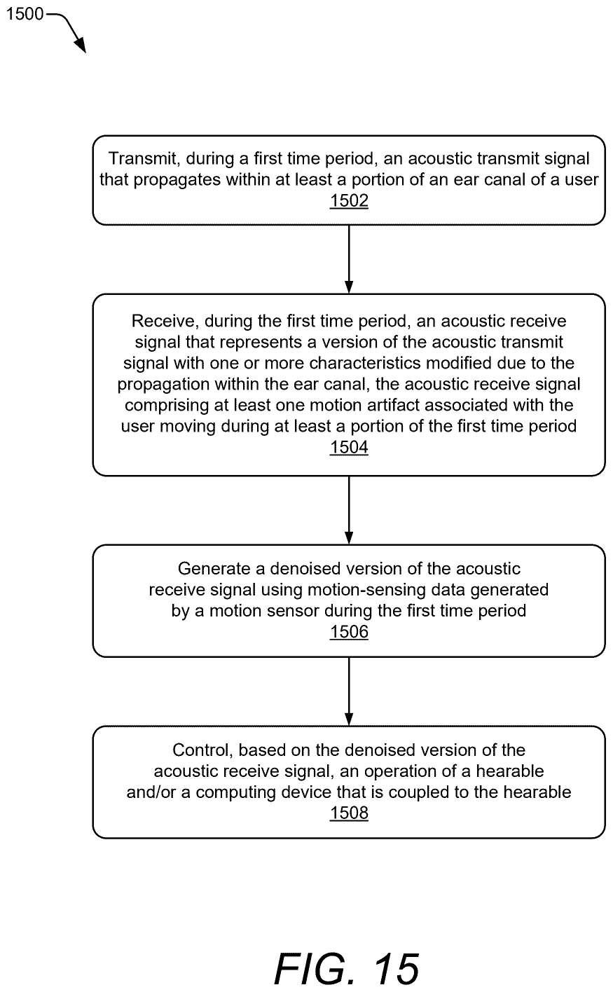

At 1502 in FIG. 15, an acoustic transmit signal that propagates within at least a portion of an ear canal of a user is transmitted during a first time period. For example, the hearable 102 transmits, during a first time period, acoustic transmit signal 602. The acoustic transmit signal 602 propagates within at least a portion of the ear canal 122 of the user 106, as shown in FIG. 6.

At 1504, an acoustic receive signal that represents a version of the acoustic transmit signal with one or more characteristics modified based on the propagation within the ear canal is received during the first time period. The acoustic receive signal comprises at least one motion artifact associated with the user moving during at least a portion of the first time period.

For example, the hearable 102 receives, during the first time period, the acoustic receive signal 604. The acoustic receive signal 604 represents a version of the acoustic transmit signal 602 with one or more characteristics modified based on the propagation within the ear canal 122. The one or more modified characteristics can include amplitude, phase, and/or frequency. The acoustic receive signal 604 also includes at least one motion artifact 312 associated with the user 106 moving during at least a portion of the first time period. The motion artifact 312 can represent a portion of the acoustic receive signal 604 where an amplitude, phase, and/or frequency is impacted by the user 106's movement.

In some cases, single-ear audioplethysmography 110 can be used to transmit and receive the acoustic signal using at least one of the hearables 102-1 or 102-2 of FIG. 6. In other cases, two-ear audioplethysmography 110 can be used to transmit the acoustic signal using the hearable 102-1 and receive the acoustic signal using the hearable 102-2.

At 1506, a denoised version of the acoustic receive signal is generated using motion-sensing data generated by a motion sensor during the first time period. For example, the motion-artifact filter 1008 generates the denoised signal 1022 using the motion-sensing data 314. The motion sensor 204 generates the motion-sensing data 314 during the first time period. The motion-artifact filter 1008 can utilize various techniques, such as informed filtering, adaptive filtering, or blind-source separation to generate the denoised signal 1022 based on the motion-sensing data 314, as described with respect to FIGS. 11-1 and 11-2. The denoised signal 1022 represents a version of the acoustic receive signal 604 in which the motion artifact 312 is attenuated (e.g., significantly attenuated or attenuated by at least three decibels) relative to the motion artifact 312 within the acoustic receive signal 604.

At 1508, an operation of a hearable and/or a computing device that is coupled to the hearable is controlled based on the denoised version of the acoustic receive signal. For example, the audioplethysmography module 1010 can generate the audioplethysmography data 712 based on the denoised signal 1022. The audioplethysmography data 712 can be used to control an operation of the hearable 102 and/or an operation of the computing device 104. Consider an example in which the audioplethysmography module 1010 performs biometric monitoring 112. In this case, the audioplethysmography module 1010 analyzes the denoised signal 1022 to measure one or more biometrics of the user 106. Example biometrics can include the user 106's heart rate, heart-rate variability, respiration rate, and/or blood pressure. An operation of the hearable 102 and/or the computing device 104 can monitor the biometric and/or communicate the biometric to the user 106.

In another example, the audioplethysmography module 1010 performs speech detection 114. By analyzing the denoised signal 1022, the audioplethysmography module 1010 can analyze the denoised signal 1022 to determine whether or not the user 106 spoke during the first time period. If the user 106's voice is detected, the hearable 102 and/or the computing device 104 can enable the voice-control interface and/or provide multi-factor voice authentication. If the user 106's voice is not detected, the hearable 102 and/or the computing device 104 can disable the voice-control interface and/or disable voice authentication.

Consider another example in which the audioplethysmography module 1010 performs chewing detection 116. In particular, the audioplethysmography module 1010 analyzes the denoised signal 1022 to determine that the user 106 is grinding their teeth. The hearable 102 and/or the computing device 104 can monitor how often bruxism is occurring, communicate this information to the user 106, and/or sound an alarm to notify the user 106.

In yet another example, the audioplethysmography module 1010 performs gesture recognition 118. In this case, the audioplethysmography module 1010 analyzes the denoised signal 1022 to recognize a gesture performed by the user 106. The recognized gesture can be mapped to a particular control of the hearable 102 and/or the computing device 104. Example controls can include changing a volume, advancing a playlist, changing content that is displayed via the computing device 104, enabling or disabling voice controls, and so forth.

Throughout the disclosure, the term “version of a signal” is used to indicate that a second signal can be a modified version of a first signal. The “version of the signal” (or the second signal) can represent a digital version of the first signal, an analog version of the first signal, an electrical version of the first signal having a voltage and current, an acoustic version of the first signal having acoustic properties, a downconverted version of the first signal having a lower range of frequencies, an upconverted version of the signal having a higher range of frequencies, a pre-processed version of the signal (e.g., a version whose amplitude, phase, and/or frequency has been modified in some way), a filtered version of the signal, and so forth. In general, the “version of the signal” represents a signal that has been modified using techniques known in the art to facilitate an operation of the hearable 102.

### Example Computing System

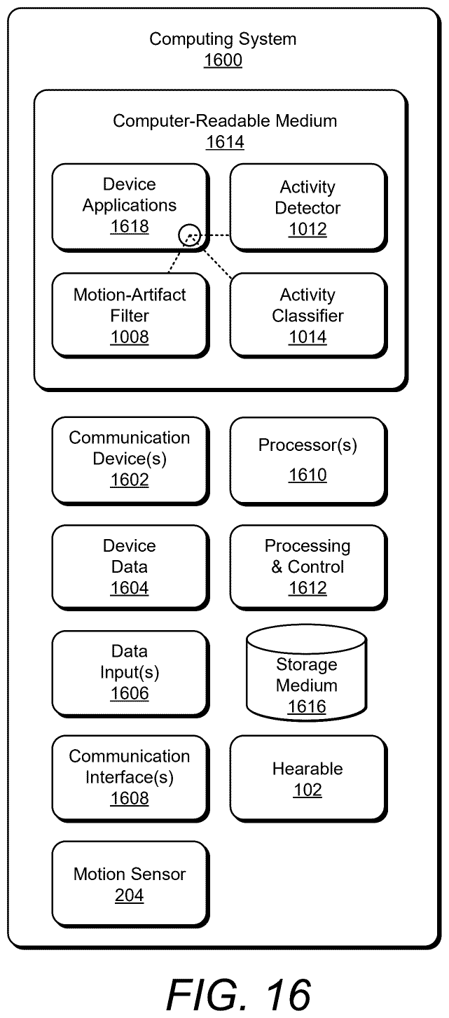

FIG. 16 illustrates various components of an example computing system 1600 that can be implemented as any type of client, server, and/or computing device as described with reference to the previous FIGS. 4 and 5 to implement aspects of audioplethysmography and motion-sensing data fusion 202.

The computing system 1600 includes communication devices 1602 that enable wired and/or wireless communication of device data 1604 (e.g., received data, data that is being received, data scheduled for broadcast, or data packets of the data). The communication devices 1602 or the computing system 1600 can include one or more hearables 102 and at least one motion sensor 204. The device data 1604 or other device content can include configuration settings of the device, media content stored on the device, and/or information associated with a user of the device. Media content stored on the computing system 1600 can include any type of audio, video, and/or image data. The computing system 1600 includes one or more data inputs 1606 via which any type of data, media content, and/or inputs can be received, such as human utterances, user-selectable inputs (explicit or implicit), messages, music, television media content, recorded video content, and any other type of audio, video, and/or image data received from any content and/or data source.

The computing system 1600 also includes communication interfaces 1608, which can be implemented as any one or more of a serial and/or parallel interface, a wireless interface, any type of network interface, a modem, and as any other type of communication interface. The communication interfaces 1608 provide a connection and/or communication links between the computing system 1600 and a communication network by which other electronic, computing, and communication devices communicate data with the computing system 1600.

The computing system 1600 includes one or more processors 1610 (e.g., any of microprocessors, controllers, and the like), which process various computer-executable instructions to control the operation of the computing system 1600. Alternatively or in addition, the computing system 1600 can be implemented with any one or combination of hardware, firmware, or fixed logic circuitry that is implemented in connection with processing and control circuits which are generally identified at 1612. Although not shown, the computing system 1600 can include a system bus or data transfer system that couples the various components within the device. A system bus can include any one or combination of different bus structures, such as a memory bus or memory controller, a peripheral bus, a universal serial bus, and/or a processor or local bus that utilizes any of a variety of bus architectures.

The computing system 1600 also includes a computer-readable medium 1614, such as one or more memory devices that enable persistent and/or non-transitory data storage (i.e., in contrast to mere signal transmission), examples of which include random access memory (RAM), non-volatile memory (e.g., any one or more of a read-only memory (ROM), flash memory. EPROM, EEPROM, etc.), and a disk storage device. The disk storage device may be implemented as any type of magnetic or optical storage device, such as a hard disk drive, a recordable and/or rewriteable compact disc (CD), any type of a digital versatile disc (DVD), and the like. The computing system 1600 can also include a mass storage medium device (storage medium) 1616.

The computer-readable medium 1614 provides data storage mechanisms to store the device data 1604, as well as various device applications 1618 and any other types of information and/or data related to operational aspects of the computing system 1600. For example, an operating system can be maintained as a computer application with the computer-readable medium 1614 and executed on the processors 1610. The device applications 1618 may include a device manager, such as any form of a control application, software application, signal-processing and control module, code that is native to a particular device, a hardware abstraction layer for a particular device, and so on.

The device applications 1618 also include any system components, engines, or managers to implement audioplethysmography and motion-sensing data fusion 202. In this example, the device applications 1618 include the motion-artifact filter 1008, the activity detector 1012, and the activity classifier 1014 of FIG. 10. Although not explicitly shown, the device applications 1618 can also include the audioplethysmography-based application 406 (APG-based application 406) of FIG. 4.

Throughout this disclosure, examples are described where a computing system 1600 (e.g., the hearable 102, the computing device 104, a client device, a server device, a computer, or another type of computing system) may analyze information (e.g., various audible and/or ultrasound signals) associated with a user, for example, the vocalization 212 mentioned with respect to FIG. 2. Further to the descriptions above, a user 106 may be provided with controls allowing the user 106 to make an election as to both if and when systems, programs, and/or features described herein may enable collection of information (e.g., information about a user's social network, social actions, social activities, profession, a user's preferences, a user's current location), and if the user is sent content or communications from a server. The computing system 1600 can be configured to only use the information after the computing system 1600 receives explicit permission from the user 106 to use the data. For example, in situations where the hearable 102 analyzes signals for biometric monitoring 112, speech detection 114, chewing detection 116, gesture recognition 118, activity detection 208 and/or activity classification 210, individual users 106 may be provided with an opportunity to provide input to control whether programs or features of the computing system 1600 can collect and make use of the data. Further, individual users 106 may have constant control over what programs can or cannot do with the information.

In addition, information collected may be pre-treated in one or more ways before it is transferred, stored, or otherwise used, so that personally-identifiable information is removed. For example, before the computing system 1600 shares data with another device, a user 106's identity may be treated so that no personally identifiable information can be determined for the user 106. Thus, the user 106 may have control over whether information is collected about the user 106 and the user 106's device, and how such information, if collected, may be used by the computing system 1600 and/or a remote computing system.

### CONCLUSION

Although techniques using, and apparatuses including, audioplethysmography and motion-sensing data fusion have been described in language specific to features and/or methods, it is to be understood that the subject of the appended claims is not necessarily limited to the specific features or methods described. Rather, the specific features and methods are disclosed as example implementations of audioplethysmography and motion-sensing data fusion.

Some examples are provided below.

Example 1: A method comprising:
transmitting and receiving, during a first time period, an acoustic signal that propagates within at least a portion of an ear canal of a user, the received acoustic signal representing a version of the transmitted acoustic signal with one or more characteristics modified based on the propagation within the ear canal, the received acoustic signal comprising at least one motion artifact associated with the user moving during at least a portion of the first time period;
accepting motion-sensing data generated by a motion sensor during the first time period;
processing a version of the received acoustic signal based on the motion-sensing data; and
generating, based on the processing, data associated with at least one of the following: the one or more characteristics associated with the propagation of the acoustic signal within the ear canal or the at least one motion artifact associated with the user moving.

Example 2: The method of example 1, wherein:
the processing of the version of the received acoustic signal comprises generating a denoised signal by filtering the version of the received acoustic signal based on the motion-sensing data, the denoised signal having motion artifacts that are attenuated relative to the motion artifacts within the version of the received acoustic signal, the denoised signal comprising the one or more characteristics; and
the generating of the data comprises generating the data based on the denoised signals.

Example 3: The method of example 2, wherein the generating of the denoised comprises:
performing informed filtering to attenuate the motion artifacts within the version of the received acoustic signal that are also present in the motion-sensing data; or
performing adaptive filtering with the received acoustic signal representing a primary reference and the motion-sensing data representing a noise reference.

Example 4: The method of example 2, wherein:
the transmitting and the receiving of the acoustic signal during the first time period comprises:
   transmitting and receiving a first acoustic signal using a first hearable; and
   transmitting and receiving a second acoustic signal using a second hearable; and
the generating of the denoised signal comprises performing adaptive filtering or blind-source separation based on a version of the received first acoustic signal, a version of the received second acoustic signal, and the motion-sensing data to generate the denoised signal and a motion signal comprising the at least one motion artifact.

Example 5: The method of example 2, wherein:
the transmitting and the receiving of the acoustic signal during the first time period comprises:
   transmitting and receiving a first acoustic signal using a first hearable; and
   transmitting and receiving a second acoustic signal using a second hearable; and
the accepting of the motion-sensing data comprises:
   accepting first motion-sensing data generated by a first motion sensor of the first hearable during the first time period; and
   accepting second motion-sensing data generated by a second motion sensor of the second hearable during the first time period:
the generating of the denoised signal comprises:
   generating a first denoised signal based on the first acoustic signal and the first motion-sensing data; and
   generating a second denoised signal based on the second acoustic signal and the second motion-sensing data; and
the generating of the data comprises generating the data based on the first denoised signal and the second denoised signal.

Example 6: The method of example 2, wherein:
the transmitting and the receiving of the acoustic signal during the first time period comprises:
   transmitting and receiving a first acoustic signal using a first hearable; and
   transmitting and receiving a second acoustic signal using a second hearable; and
the generating of the denoised signal comprises providing a version of the received first acoustic signal, a version of the received second acoustic signal, and the motion-sensing data as inputs to a machine-learned model to generate the denoised signal and a motion signal.

Example 7: The method of any one of examples 2 to 6, wherein the generating of the data comprises measuring a biometric of the user based on the denoised signal.

Example 8: The method of any previous example, wherein the processing of the version of the received acoustic signal comprises determining that the user moved during the portion of the first time period based on a correlation between the at least one motion artifact within the received acoustic signal and the motion-sensing data.

Example 9: The method of any previous example, wherein the processing of the version of the received acoustic signal comprises classifying, based on the at least one motion artifact within the received acoustic signal and the motion-sensing data, a type of activity that the user performed during the first time period.

Example 10: The method of example 9, wherein the classifying of the type of activity comprises determining that the type of activity is relatively motionless or associated with a substantial amount of motion.

Example 11: The method of any one of examples 8 to 10, further comprising:
controlling an operation of a hearable or an operation of a computing device that is coupled to the hearable based on at least one of the following:
   a determination that the user moved; or
   a classification of the type of activity that the user performed.

Example 12: The method of any previous example, wherein the motion-sensing data comprises at least one of the following:
linear accelerations associated with three orthogonal axes; or
rotational velocities associated with the three orthogonal axes.

Example 13: The method of any previous example, further comprising:
transmitting and receiving, prior to the first time period, another acoustic signal that propagates within at least the portion of the ear canal of the user, the other acoustic signal having multiple tones, the received other acoustic signal representing a version of the transmitted other acoustic signal with one or more characteristics modified due to the propagation within the ear canal; and
selecting a subset of the multiple tones based on the received other acoustic signal,
wherein the transmitting and the receiving of the acoustic signal during the first time period comprises transmitting and receiving the acoustic signal having the subset of the multiple tones.

Example 14: The method of any previous example, wherein the acoustic signal comprises an ultrasound signal having frequencies between approximately twenty and ninety-six kilohertz.

Example 15: The method of any previous example, further comprising:
transmitting audible content during at least the portion of the first time period.

Example 16: The method of any previous example, wherein the generated data is used to control an operation of a hearable and/or a computing device.

Example 17: The method of example 16, wherein the generated data is used to customize a measurement of biometrics of the user.

Example 18: The method of example 17, wherein the generated data is used to determine whether to tune a filter for detecting a heart rate of the user and/or a respiration rate of the user based on the received acoustic signal.

Example 19: A computer-readable storage medium comprising instructions that, responsive to execution by a processor, cause a hearable to perform any one of the methods of examples 1 to 18.

Example 20: A device comprising:
at least one transducer; and
at least one processor,
the device configured to perform, using the at least one transducer and the at least one processor, any one of the methods of examples 1 to 18.

Example 21: The device of example 20, further comprising:
a speaker; and
an active-noise-cancellation circuit comprising a feedback microphone,
wherein the at least one transducer comprises the speaker and the feedback microphone.

Example 22: The device of example 20, wherein:
the at least one transducer comprises a speaker and a microphone;
the speaker is configured to be positioned proximate to a first ear of a user; and
the microphone is configured to be positioned proximate to a second ear of the user.

Example 23: The device of any one of examples 20 to 22, further comprising a motion sensor.

Example 24: The device of any one of examples 20 to 23, wherein the device comprises at least one earbud.

Example 25: A method comprising:
transmitting, during a first time period, an acoustic transmit signal that propagates within at least a portion of an ear canal of a user;
receiving, during the first time period, an acoustic receive signal that represents a version of the acoustic transmit signal with one or more characteristics modified due to the propagation within the ear canal, the acoustic receive signal comprising at least one motion artifact associated with the user moving during at least a portion of the first time period;
generating a denoised version of the acoustic receive signal using motion-sensing data generated by a motion sensor during the first time period, the at least one motion artifact within the denoised version of the acoustic receive signal being attenuated relative to the at least one motion artifact within the acoustic receive signal; and
controlling, based on the denoised version of the acoustic receive signal, an operation of a hearable and/or a computing device that is coupled to the hearable.

Example 26: The method of example 25, further comprising:
measuring at least one biometric of the user based on the denoised version of the acoustic receive signal,
wherein the controlling the operation of the hearable and/or the computing device comprises communicating the at least one biometric.

Example 27: The method of example 26, wherein the at least one biometric comprises at least one of the following:
a heart rate of the user;
heart-rate variability of the user;
a respiration rate of the user; or
blood pressure.

Example 28: The method of any previous example, further comprising:
determining that the user spoke during the first time period based on the denoised signal,
wherein the controlling the operation of the hearable and/or the computing device comprises at least one of the following:
   enabling a voice-control interface based on the determination; or
   providing multi-factor voice authentication based on the determination;

Example 29: The method of any previous example, further comprising:
recognizing a gesture performed by the user during the first time period based on the denoised version of the acoustic receive signal,
wherein the controlling the operation of the hearable and/or the computing device comprises controlling operation of the hearable and/or the computing device based on the recognized gesture.

Example 30: The method of any previous example, further comprising:
determining that the user is grinding their teeth during the first time period based on the denoised version of the acoustic receive signal,
wherein the controlling the operation of the hearable and/or the computing device comprises controlling the operation of the hearable and/or the computing device based on the determination.

Example 31: The method of any previous example, wherein the generating of the denoised version of the acoustic receive signal comprises tuning a passband of a filter based on the motion-sensing data to cause the filter to pass frequencies of the version of the acoustic receive signal that correspond to biometric monitoring, speech detection, chewing detection, and/or gesture recognition.

Example 32: The method of any one of examples 25 to 30, wherein the generating of the denoised version of the acoustic receive signal comprises performing adaptive filtering with the acoustic receive signal representing a primary reference and the motion-sensing data representing a noise reference.

Example 33: The method of any previous example, further comprising:
transmitting, during the first time period, a second acoustic transmit signal that propagates within at least a portion of a second ear canal of the user; and
receiving, during the first time period, a second acoustic receive signal that represents a version of the second acoustic transmit signal with one or more characteristics modified due to the propagation within the second ear canal, the second acoustic receive signal comprising at least one second motion artifact associated with the user moving during at least the portion of the first time period,
wherein the generating of the denoised version of the acoustic receive signal comprises:
   performing adaptive filtering or blind-source separation using the version of the acoustic receive signal, a version of the second acoustic receive signal, and the motion-sensing data to generate the denoised signal and a motion signal comprising the at least one motion artifact.

Example 34: The method of example 33, further comprising:
determining that the user moved during at least the portion of the first time period based on the motion signal.

Example 35: The method of example 33 or 34, further comprising:
classifying, based on the motion signal, a type of activity that the user performed during the first time period.

Example 36: The method of example 35, wherein the classifying of the type of activity comprises determining that the type of activity is relatively motionless or associated with a substantial amount of motion.

Example 37: The method of any one of examples 25 to 32, further comprising:
transmitting, during the first time period, a second acoustic transmit signal that propagates within at least a portion of a second ear canal of the user; and
receiving, during the first time period, a second acoustic receive signal that represents a version of the second acoustic transmit signal with one or more characteristics modified due to the propagation within the second ear canal, the second acoustic receive signal comprising at least one second motion artifact associated with the user moving during at least the portion of the first time period;
generating a denoised version of the second acoustic receive signal using other motion-sensing data generated by a second motion sensor during the first time period,
wherein the controlling of the operation of the hearable and/or the computing device comprises controlling the operation of the hearable and/or the computing device based on the denoised versions of the acoustic receive signal and the second acoustic receive signal.

Example 38: The method of any one of examples 25 to 37, wherein the motion-sensing data comprises at least one of the following:
linear accelerations associated with three orthogonal axes; or
rotational velocities associated with the three orthogonal axes.

Example 39: The method of any one of examples 25 to 38, further comprising:
transmitting, prior to the first time period, another acoustic transmit signal that propagates within at least the portion of the ear canal of the user, the other acoustic transmit signal having multiple tones;
receiving, prior to the first time period, another acoustic receive signal representing a version of the other acoustic transmit signal with one or more characteristics modified due to the propagation within the ear canal; and
selecting a subset of the multiple tones based on quality metrics associated with the multiple tones,
wherein the transmitting of the acoustic transmit signal during the first time period comprises transmitting the acoustic transmit signal having the subset of the multiple tones.

Example 40: The method of any one of examples 25 to 39, wherein the acoustic transmit signal comprises an ultrasound signal having frequencies between approximately twenty and ninety-six kilohertz.

Example 41: The method of any previous example, further comprising:
transmitting audible content during at least the portion of the first time period.

Example 42: A computer-readable storage medium comprising instructions that, responsive to execution by a processor, cause a hearable to perform any one of the methods of examples 25 to 41.

Example 43: A device comprising:
at least one transducer; and
at least one processor,
the device configured to perform, using the at least one transducer and the at least one processor, any one of the methods of examples 25-41.

Example 44: The device of example 43, further comprising:
a speaker; and
an active-noise-cancellation circuit comprising a feedback microphone,
wherein the at least one transducer comprises the speaker and the feedback microphone.

Example 45: The device of example 43, wherein:
the at least one transducer comprises a speaker and a microphone;
the speaker is configured to be positioned proximate to a first ear of a user; and
the microphone is configured to be positioned proximate to a second ear of the user.

Example 46: The device of any one of examples 43 to 45, further comprising a motion sensor.

Example 47: The device of any one of examples 43 to 46, wherein the device comprises at least one earbud.

What is claimed is:

1. A method comprising:
transmitting and receiving, during a first time period, an acoustic signal that propagates within at least a portion of an ear canal of a user, the received acoustic signal representing a version of the transmitted acoustic signal with one or more characteristics modified based on the propagation within the ear canal, the received acoustic signal comprising at least one motion artifact associated with the user moving during at least a portion of the first time period;
accepting motion-sensing data generated by a motion sensor during the first time period;
processing a version of the received acoustic signal based on the motion-sensing data; and
generating, based on the processing, data associated with at least one of the following: the one or more characteristics associated with the propagation of the acoustic signal within the ear canal or the at least one motion artifact associated with the user moving.

2. The method of claim 1, wherein:
the processing of the version of the received acoustic signal comprises generating a denoised signal by filtering the version of the received acoustic signal based on the motion-sensing data, the denoised signal having motion artifacts that are attenuated relative to the motion artifacts within the version of the received acoustic signal, the denoised signal comprising the one or more characteristics; and
the generating of the data comprises generating the data based on the denoised signal.

3. The method of claim 2, wherein the generating of the denoised signal comprises:
performing informed filtering to attenuate the motion artifacts within the version of the received acoustic signal that are also present in the motion-sensing data; or
performing adaptive filtering with the received acoustic signal representing a primary reference and the motion-sensing data representing a noise reference.

4. The method of claim 2, wherein:
the transmitting and the receiving of the acoustic signal during the first time period comprises:
transmitting and receiving a first acoustic signal using a first hearable; and
transmitting and receiving a second acoustic signal using a second hearable; and
the generating of the denoised signal comprises performing adaptive filtering or blind-source separation based on a version of the received first acoustic signal, a version of the received second acoustic signal, and the motion-sensing data to generate the denoised signal and a motion signal comprising the at least one motion artifact.

5. The method of claim 2, wherein:
the transmitting and the receiving of the acoustic signal during the first time period comprises:
transmitting and receiving a first acoustic signal using a first hearable; and
transmitting and receiving a second acoustic signal using a second hearable; and
the accepting of the motion-sensing data comprises:
accepting first motion-sensing data generated by a first motion sensor of the first hearable during the first time period; and
accepting second motion-sensing data generated by a second motion sensor of the second hearable during the first time period;
the generating of the denoised signal comprises:
generating a first denoised signal based on the first acoustic signal and the first motion-sensing data; and
generating a second denoised signal based on the second acoustic signal and the second motion-sensing data; and
the generating of the data comprises generating the data based on the first denoised signal and the second denoised signal.

6. The method of claim 2, wherein:
the transmitting and the receiving of the acoustic signal during the first time period comprises:
transmitting and receiving a first acoustic signal using a first hearable; and
transmitting and receiving a second acoustic signal using a second hearable; and
the generating of the denoised signal comprises providing a version of the received first acoustic signal, a version of the received second acoustic signal, and the motion-sensing data as inputs to a machine-learned model to generate the denoised signal and a motion signal.

7. The method of claim 2, wherein the generating of the data comprises measuring a biometric of the user based on the denoised signal.

8. The method of claim 1, wherein the processing of the version of the received acoustic signal comprises determining that the user moved during the portion of the first time period based on a correlation between the at least one motion artifact within the received acoustic signal and the motion-sensing data.

9. The method of claim 1, wherein the processing of the version of the received acoustic signal comprises classifying, based on the at least one motion artifact within the received acoustic signal and the motion-sensing data, a type of activity that the user performed during the first time period.

10. The method of claim 9, wherein the classifying of the type of activity comprises determining that the type of activity is relatively motionless or associated with a substantial amount of motion.

11. The method of claim 8, further comprising:
controlling an operation of a hearable or an operation of a computing device that is coupled to the hearable based on at least one of the following:
a determination that the user moved; or
a classification of the type of activity that the user performed.

12. The method of claim 1, wherein the motion-sensing data comprises at least one of the following:
linear accelerations associated with three orthogonal axes; or
rotational velocities associated with the three orthogonal axes.

13. The method of claim 1, further comprising:
transmitting and receiving, prior to the first time period, another acoustic signal that propagates within at least the portion of the ear canal of the user, the other acoustic signal having multiple tones, the received other acoustic signal representing a version of the transmitted other acoustic signal with one or more characteristics modified due to the propagation within the ear canal; and
selecting a subset of the multiple tones based on the received other acoustic signal,
wherein the transmitting and the receiving of the acoustic signal during the first time period comprises transmitting and receiving the acoustic signal having the subset of the multiple tones.

14. The method of claim 1, wherein the generated data is used to control an operation of a hearable and/or a computing device.

15. The method of claim 14, wherein the generated data is used to customize a measurement of biometrics of the user.

16. A computer-readable storage medium comprising instructions that, responsive to execution by a processor, cause a hearable to:
transmit and receive, during a first time period, an acoustic signal that propagates within at least a portion of an ear canal of a user, the received acoustic signal representing a version of the transmitted acoustic signal with one or more characteristics modified based on the propagation within the ear canal, the received acoustic signal comprising at least one motion artifact associated with the user moving during at least a portion of the first time period;
accept motion-sensing data generated by a motion sensor during the first time period;
process a version of the received acoustic signal based on the motion-sensing data; and
generate, based on the processing, data associated with at least one of the following: the one or more characteristics associated with the propagation of the acoustic signal within the ear canal or the at least one motion artifact associated with the user moving.

17. A device comprising:
at least one transducer configured to transmit and receive, during a first time period, an acoustic signal that propagates within at least a portion of an ear canal of a user, the received acoustic signal representing a version of the transmitted acoustic signal with one or more characteristics modified based on the propagation within the ear canal, the received acoustic signal comprising at least one motion artifact associated with the user moving during at least a portion of the first time period; and
at least one processor configured to:
accept motion-sensing data generated by a motion sensor during the first time period;
process a version of the received acoustic signal based on the motion-sensing data; and
generate, based on the processing, data associated with at least one of the following: the one or more characteristics associated with the propagation of the acoustic signal within the ear canal or the at least one motion artifact associated with the user moving.

18. The device of claim 17, further comprising:
a speaker; and
an active-noise-cancellation circuit comprising a feedback microphone,
wherein the at least one transducer comprises the speaker and the feedback microphone.

19. The device of claim 17, wherein:
the at least one transducer comprises a speaker and a microphone;
the speaker is configured to be positioned proximate to a first ear of a user; and
the microphone is configured to be positioned proximate to a second ear of the user.

20. The device of any one of claim 17, further comprising a motion sensor.
